(19) United States
(12) Patent Application Publication
(10) Pub. No.: US 2022/0364033 A1
(43) Pub. Date: Nov. 17, 2022

(54) EMBEDDING BATH

(71) Applicant: UNIVERSITEIT TWENTE, Enschede (NL)

(72) Inventors: Claas Willem VISSER, Enschede (NL); Vasileios TRIKALITIS, Enschede (NL); Jeroen ROUWKEMA, Enschede (NL); Tom KAMPERMAN, Enschede (NL)

(73) Assignee: UNIVERSITEIT TWENTE, Enschede (NL)

(21) Appl. No.: 17/767,298

(22) PCT Filed: Oct. 9, 2020
(86) PCT No.: PCT/NL2020/050625

§ 371 Date: Apr. 7, 2022

(30) Foreign Application Priority Data
Oct. 11, 2019 (NL) 2023996

(51) Int. Cl. C12M 1/12; A61L 27/38; C12N 5/00; C12M 3/00; A61L 27/50

(52) U.S. Cl. CPC C12M 25/16; A61L 27/38; C12N 5/0062; C12M 21/08; A61L 27/50; B33Y 10/00

(57) ABSTRACT

The invention provides a construct (1) comprising a number N of material types (100, 110, . . . ), wherein N is at least 2, wherein at least two of the material types (100, 110, . . . ) comprise granular material (101) comprising particles (10), wherein the granular material (101) at least defines an exterior surface (6) of the construct (1), wherein the construct (1) is self-supporting, and wherein the construct (1) is (i) self-healing or is (ii) configured for being self-healing by changing a liquid (15) content of the construct (1); wherein the different material types (100, 110, . . . ) mutually differ in at least one characteristic (19) selected from the group consisting of a physical characteristic and a chemical characteristic.

### FIELD OF THE INVENTION

The invention relates to an embedding bath for tissue engineering, a method for producing such embedding bath and a method for the manufacture of engineered tissue applying the embedding bath.

### BACKGROUND OF THE INVENTION

Embedded baths and processes for the production of embedded baths are known in the art. In Science Advances, 23 Oct. 2015:Vol. 1(9), e1500758, Hinton et al., “Three-dimensional printing of complex biological structures by freeform reversible embedding of suspended hydrogels”, e.g., the authors describe the additive manufacturing of complex three-dimensional (3D) biological structures using soft protein and polysaccharide hydrogels that are challenging or impossible to create using traditional fabrication approaches. The structures are built by embedding the printed hydrogel within a secondary hydrogel that serves as a temporary, thermo reversible, and biocompatible support. The process enables 3D printing of hydrated materials with an elastic modulus <500 kPa including alginate, collagen, and fibrin. In a review article in Current Opinion in Biotechnology 2019, 60: 1-8, Riley et al., Granular hydrogels: emergent properties of jammed hydrogel microparticles and their applications in tissue repair and regeneration the authors describe that granular hydrogels are emerging as a versatile and effective platform for tissue engineered constructs in regenerative medicine. The hydrogel microparticles (HMPs) that compose these materials exhibit particle jamming above a minimum packing fraction, which results in a bulk, yet dynamic, granular hydrogel scaffold. Recently, they have been utilized as cell cultures platforms and extracellular matrix mimics with remarkable success in promoting cellular infiltration and subsequent tissue remodeling in vivo. WO2016182969 describes a three-dimensional cell growth medium that may comprise hydrogel particles swollen with a liquid cell growth medium to form a granular gel yield stress material which undergoes a phase transformation from a solid phase to a liquid-like phase when an applied stress exceeds the yield stress. Cells may be placed in the three-dimensional cell growth medium according to any shape or geometry, and may remain in place within the three-dimensional cell growth medium. Further, WO2017049066 describes a biological cell and/or tissue growth apparatus operable to create, in a chamber of the apparatus, a three-dimensional (3D) cell culture and to interact with a 3D structure of the cells in the chamber. The apparatus may include equipment for printing the 3D cell culture in a 3D cell growth medium. The 3D cell growth medium may be a granular gel material that undergoes a temporary phase change in response to an applied stress. The apparatus may be operated such that the 3D printing equipment “prints” the 3D cell culture by depositing cells at particular locations in the 3D cell growth medium. Further, Bhattacharjee et al., Science Advances Vol. 1(8), e1500655, “Writing in the granular medium” describe gels made from soft microscale particles that smoothly transition between the fluid and solids states. The authors describe three-dimensional structures that are created in the medium by injecting material in the granular medium along a spatial path.

### SUMMARY OF THE INVENTION

In tissue engineering a material may be extruded in a second material or a “support bath”. The injected material may thus provide at least part of the tissue to be engineered. The second material may function as a temporary support, embedding and (locally) supporting (and keeping in place) the injected material and e.g. allowing the tissue to develop. Because of its function, the second material or support bath (optionally in a mold) may also be referred to as “an embedding (printing) bath”. The embedding bath is especially configured for allowing the first material to be extruded, injected or e.g. deposited in the second material (or embedding bath) (at a predefined position) and for supporting the first material substantially at the predefined position where it is deposited (in the embedding bath).

Natural tissue has a spatiotemporally defined environmental structure which preferably is also provided in engineered tissue. However, bio fabrication methods known in the art using embedding baths to produce the tissue seem not to be able to replicate the natural extracellular structure of cells at the time of fabrication and may not function as a cell or tissue instructive material. Spatiotemporal control of the cell and tissue microenvironment may however be very relevant in order to achieve natural tissue analogues. Current production methods for producing embedding baths are typically based on cutting of material into small particles, polymer hydration or cooling while centrifuging. This may create limitation in either particle size and shape control and material composition. Furthermore, embedding baths are currently made from a single material phase which may frustrate the desired spatiotemporal control. Specifically, known embedding baths are homogeneous granular media that do not contain different spatially defined or compartmentalized regions of granular media. In addition to that, present methods may use (photo) crosslinking or solidifying mechanisms that involve chemical reactions, including radical-based reactions, that may be detrimental to biological cells. The ability to not have the cells subjected to these processes seems desirable. Furthermore, the ability to create new spatially or temporally controlled chemically, physically, or biologically instructive cues in the microenvironment, or to combine these cues with pre-existing ones is desirable but yet unattainable.

Current methods for embedding baths may further involve production in situ of the experiment. Implicating that each laboratory using an embedding bath has to acquire technological know-how and fine tune their production methods before they may actually be able to three-dimensional (3D) print in the embedding bath. The embedding bath kits in the market and also the research approaches currently implemented, can only create microparticle baths with a single composition. Furthermore current particle baths are not functional in terms of biological phenomena, but merely act as a mechanical support. It is furthermore observed that current embedding baths are not reproducibly made: every lab uses slightly different protocols and printers. There is a need for standardized 3D embedded baths, and especially embedding baths with additional functions, next to the supporting function.

Hence, it is an aspect of the invention to provide an alternative embedding (printing) bath or construct, which preferably further at least partly obviates one or more of above-described drawbacks. It is a further aspect to provide a method for producing such construct/embedding bath, which preferably further at least partly obviates one or more of above-described drawbacks. The invention further provides a method for manufacturing of engineered tissue which preferably further at least partly obviates one or more of above-described drawbacks. The present invention may have as object to overcome or ameliorate at least one of the disadvantages of the prior art, or to provide a useful alternative.

The construct/embedding bath of the invention may be designed according to need. The embedding bath may provide a high degree of spatial and/or temporal control and may be used to manufacture (biological/cell-based) tissues with complex geometries. The embedding bath especially comprises spatial and/or temporal stimuli (or “cues”) that (3D printed) targeted further material may interact with. The embedding bath may be produced in a reproducible way and may provide standardized 3D cell culture methods, 3D cell-based tissue growth (i.e., ‘tissue engineering’) methods, or 3D cell or tissue analysis methods. Moreover, the embedding baths may comprise particles reversibly annealed (or firmly attracted or attached) to each other without destroying the spatial and/or temporal information or biological cells in it. Annealing or keeping the particles together may be based on a physical and/or chemical interaction without the need for mechanisms that may be detrimental to cells. Annealing of particles may for example be entirely or partially based on bio-orthogonal chemistry that induces or enables interaction between the particles of which the granular medium is made of but does not chemically or physically affect biological matter such as the cells or tissue that may be deposited within the embedding bath. The embedding baths may allow extraction of liquid biopsies from tissue provided/growing in the embedding baths. Moreover, structures consisting of multiple aggregated cells or multiple cells within extracellular matrix, liquid, particles that may contain functionalized moieties that act as anchoring sites (e.g., to enable particle/cell interactions), cell markers or fluorescent detectors, or tissue mass itself may in embodiments be extracted for the purpose of diagnostics. The construct as such may provide the stability that enables long term culture of 3D-tissue. No additional crosslinking of material of the construct may be required to obtain the required stability. The construct may allow the creation of organ on a chip models and is especially fully compatible with lab on a chip applications. The invention especially provides in embodiments 3D printed free-standing embedding baths offering spatiotemporal control for the engineering of tissues and soft matter components.

In a first aspect, the invention provides a construct (or “embedding bath”) comprising (an assembly of) a number (N) of material types, especially wherein N is at least 2. The (different) material types are especially spatially arranged in regions. The (different) material types may (thus) be configured in (or over) compartments. The (different) material types may define compartments. The construct is especially compartmentalized. The construct is especially an embedding bath (see further below). The construct is especially self-standing.

Especially, at least one of the material types comprises (or is) (a) (respective) granular material. The granular material especially comprises (respective) particles. In further embodiments, at least two of the material types comprise (respective) granular material. The granular material, especially at least part of the granular material, may in embodiments define (at least part of) an exterior surface of (the (assembly of) material types of) the construct. The construct is especially self-supporting. In further embodiments, (at least part of) the construct may be self-healing. Yet, in further embodiments, (at least part of) the construct is configured for being self-healing, especially by changing a liquid content of ((the) at least part of) the construct, especially of the granular material. The different material types may mutually differ in at least one characteristic selected from the group consisting of a physical characteristic and a chemical characteristic. Hence, at least part of the compartments may mutually differ in a physical characteristic and/or chemical characteristic (of material of (arranged in) the compartment).

The term “material type” especially relates to a subset of materials (especially a subset of materials of the construct). The material types not necessarily all comprise (a) granular material. In embodiments at least one of the material types comprises (a) non-granular material (see below).

Herein the terms “self-supporting”, “self-standing”, “self-sustaining” are used in relation to structures that may stay up or upright without being supported by something else. These terms may be used interchangeably, herein. Further, also the term “free standing” may be used to refer to “self-standing”. The term “self-standing” especially refers to being able to hold/carry/support its own weight without changing its dimensions/shape. Especially, a characteristic dimension of the construct may substantially not change during a determined time frame (see also below) after the construct is provided (e.g. according to the method described herein). The construct may support its own weight substantially without changing its shape. An aspect ratio height over width of the construct may be larger than 1. The aspect ratio of a self-standing construct may not significantly change in time. The height of the construct may in embodiments e.g. at least be ten times, such as at least hundred times or even thousand times, a number averaged size of (the) particles of the construct and/or particles of which the construct is made, while not collapsing under its own weight. Interparticle forces or forces between the particles (mutually forcing the particles towards each other) of the granular material may thus especially be larger than external forces (such as gravity) acting on the particles (and forcing the particles away from each other and especially forcing the construct to collapse under its own weight). To be self-supporting, the particles in the granular material may especially be in a jammed state (see below).

The free-standing property of the granular material may relate to a total volume of the particles in the granular material relative to a total volume of the respective granular material (i.e. a volume fraction (v_{f}) of the particles). This volume fraction may especially be selected high enough to allow printing the granular material and to provide a self-supporting (part of the) construct. The volume fraction (v_{f}) (of the self-standing construct) is especially larger than a random close packing of identical non-deformable spheres (64% v/v), such as equal to or larger than 70% v/v, even more especially larger than the densest (or maximum) packing of identical non-deformable spheres (74%), such as at least 80% (v/v), or at least 85% v/v. In embodiments, v_{f} is at least 90% v/v, such as in the range of 90-95% v/v. Further, of may especially be equal to or smaller than 95% v/v, such as equal to or smaller than 90% v/v, especially equal to or smaller than 85% v/v, such as equal to or smaller than 80% v/v, especially to provide self-standing properties to the granular material (and/or of the construct). In embodiments, v_{f} is at least 70% v/v, such as in the range of 70-90% v/v, especially in the range 70-80% v/v. The selected volume fraction relates to (physical) properties of the particles. For instance, v_{f} may be smaller for a first granular material comprising stiff particles than for a further granular material comprising less stiff particles. Also, a more heterogenous distribution of size of the particles may require a higher v_{f} to provide self-standing properties compared to a monodispersed particle distribution.

The construct may have embedding bath (or supporting bath) properties. The construct may therefore be configured for locally supporting a (targeted) further material being provided into the construct (see below). The construct is especially an embedding bath. The construct, especially the embedding bath, is especially free standing. Free standing of the embedding bath (or construct) may thus relate to the fact that the embedding bath (construct) may substantially retain its external shape (dimensions) and internal patterning (see below) when not supported by further elements (such as walls) (i.e. especially next to a base or support the embedding bath (or construct) is arranged on) over a certain time frame. Especially, the self-supporting relates to the construct also when not being contained in (supported by) a container. Such time frame may be in the range of a couple of second, or a couple of minutes, or at least a couple of hours. In embodiments, the time frame is at least 1 minute, especially at least 1 hour. In further embodiments, the embedding bath (construct) may retain its external shape and internal patterning (when not being supported by further elements) over at least 6 hours, such as at least 1 day, especially at least 1 week. The time frame may in embodiments be weeks, or even month. In the time frame, elements of the embedding bath (construct) defining the internal patterning or the external shape may not have moved more than 2 mm, especially not more than 1 mm, even more especially not more than 0.5 mm.

Especially a (characteristic) dimension of the construct may substantially not change in the time frame. The dimension may, e.g., be a height of the construct or a width of the construct. In embodiments, e.g., (during the time frame) a change in the (maximum) height of the construct (when not being supported by further elements) may be less than 10%, especially less than 5%, or even less than 1%. Further, (during the time frame) a change in the (maximum) width of the construct (when not being supported by further elements) may be less than 10%, especially less than 5%, or even less than 1%. In embodiments, the height of the (free-standing) construct at the end of the time frame, such as after a week or after a month, is at least 95% of the height of the construct at the moment it is provided (the beginning of the time frame). It should be noted that such change in dimensions is related to the construct at atmospheric conditions, especially wherein evaporation of interstitial liquid may be prevented. The change of dimensions especially relates to the construct in which the volume fraction (v_{f}) of the particles is not changed (because of evaporation) (in that time frame).

The construct may in embodiments be a precursor for an embedding bath (i.e. requiring a further action, such as adapting a volume fraction of the particles (relative to a volume of the granular material they are part of), to provide the embedding bath) or “precursor embedding bath”. In embodiments, (at least part of) the construct is self-healing when providing a (hydration) fluid to the construct. Alternatively, (at least part of) the construct may be self-healing when removing a fluid from the construct. Herein the term “embedding bath” (as well as “construct”) may also refer to such precursor embedding bath.

The construct especially comprises spatial and/or temporal information or bioactive compounds that may provide instructive cues to (biological) cells or tissues, for example, for engineering 3D tissues (for tissue to be cultured). Herein the term “spatiotemporal” is used. The term may relate to spatial and/or temporal. For instance spatiotemporal information may refer to information in relation to a (relative) (3D) location and/or information in relation to a (relative) moment in time.

It is noted that herein the terms “cell” and “biological cell” and the like may be used interchangeably. The term “cell” especially refers to a biological cell. A biological cell is the basic structural, functional, and biological unit of an organism. Cells may also be referred to as “building blocks of life”. The (biological) cell may in embodiments be a be eukaryotic cell. In further embodiments the cell may refer to a prokaryotic cell. The term “(biological) cell” may also relate to a plurality of (different) cells, e.g. a plurality of eukaryotic cells and/or a plurality of prokaryotic cells or a combination of one or more eukaryotic cells and one or more prokaryotic cells. The term may relate to a single (biological) cell, or in embodiments to over 10 million or even many more (biological cells). Moreover, the term “cell”, and especially also the term “cells”, may in embodiments refer to a cell aggregate, a spheroid (see further below), and/or an organoid. The term (biological) cells may further refer to a cell type, or to a plurality of (different) cell types. Examples of cell types are e.g. a heart cell, a lung cell, a brain cell, a skin cell, a muscle cell, a stem cell, a fibroblast, etc. (see also below).

The term “self-healing” refers to the built-in ability to automatically repair damage provided to the material. In embodiments, self-healing refers to closing of openings in the granular medium through influx of granular medium. In further embodiments, self-healing refers to closing of openings in the granular medium through expansion or swelling of particles that may have been shrunk or squeezed during the formation of a cut or damaged area. For instance, when the material is cut (damaged) with a specific object, and the object is removed again, the material may regain its original (spatial) configuration, wherein the cut is closed. Regaining its original configuration may be based on physical and/or chemical interactions of compounds of the material. In embodiments, a spontaneous formation of new interactions may occur when old interactions are broken within the material. The self-healing property of the construct may enable e.g. movement of a printing nozzle or needle (in the construct) for high resolution printing a further material, such as biological cell material, in the construct without destroying the spatial information in the construct. It may further allow to extract biopsies from the tissue cultured in the construct

It will be understood that the self-healing capacity may depend on the amount of material displaced or deformed. If a cut is made with a very thick needle, e.g., having an outer diameter of about 5 mm or more, the original configuration may not be completely restored. Yet, herein self-healing especially relates to healing of damage or cuts provided with a printing nozzle and/or needle. Such nozzle or needle may e.g. have an outer diameter being equal to or smaller than 2 mm, especially equal to or smaller than 1 mm, such as equal to or smaller than 0.5 mm. Further, the self-healing property may especially (also) relate to the volume fraction of particles in the granular material.

The volume fraction (v_{f}) (of the self-healing construct) is especially (also) larger than the random closed packing of identical non-deformable spheres (64% v/v), especially equal to or larger than 70% v/v, such as especially larger than the densest packing of identical non-deformable spheres (74%), such as at least 80% (v/v), or at least 85% v/v. In embodiments, vf is at least 90% v/v, such as in the range of 90-95% v/v. Further, v_{f} may especially be equal to or smaller than 95% v/v, such as equal to or smaller than 90%, especially equal to or smaller than 85% v/v, even more especially in embodiments equal to or smaller than 80% v/v, especially to provide self-healing properties to the granular material (and/or the construct). Hence, it will be understood that in embodiments, v_{f} may be selected to provide self-supporting and self-healing granular material, especially a self-healing self-supporting construct (embedding bath). In embodiments v_{f} in the range of 70-90% v/v, such as in the range of 74-90% v/v, especially in the range of 74-85% v/v, such as in the range of 76-82% 30 v/v. In other embodiments, v_{f} is selected in the range of 68-90% v/v, such as in the range of 70%-80% v/v. In yet further embodiments, especially comprising substantially deformable particles v_{f} is selected in the range of 70-95% v/v, especially in the range of 80-90% v/v, such as in the range of 82-88% v/v. In embodiments, the v_{f} for providing a self-standing construct may be different from (especially higher than) the v_{f} for providing a self-healing construct. In the method of the invention the v_{f} may be changed in embodiments after having provided (printed) the construct (and before depositing a further material in the construct (see further below).

Herein the volume fraction of of particles in the granular material especially refers to at least the volume fraction in the granular material defining the exterior surface. The term further especially refers to at least the granular material configured for dispensing a further material in (such as in the method for producing engineering tissue described herein)

In specific embodiments, a plurality of the material types comprise (especially are) (a respective) granular material. Hence, the term “granular material” may especially refer to a plurality of (different) types of granular material/granular materials. For instance, a first material type may comprise a first granular material, and especially a second material type may comprise a second granular material, and especially a further material type may comprise a further granular material, etc. The different granular materials may especially comprise different particles respectively, especially wherein the particle properties may differ between the granular materials. In embodiments, the particle properties of different granular materials may be substantially the same. Moreover, e.g. additionally or alternatively properties of a fluid between the particles (see below) of the first granular material may differ from properties of a further fluid between the particles of a further granular material.

The term “granular material” especially relates to a collection of discrete particles or ‘granules’ in contact. Some examples of granular materials are snow, sand, rice, salts, coffee, cereal grains, corn flakes, glass beads. Some further examples of granular materials are powders, microparticles, microgels, cells, and cell aggregates (which may also be indicated as “spheroids”). The particles or granules may comprise any arbitrarily composition and may e.g. also be hollow comprising only a shell (which may herein also be indicates by the term “capsule” or a “core-shell particle”). The particles may further comprise a liquid or be composed of one or more liquids. Especially, small particles even when being composed of liquids may have characteristics of a solid particle. Therefore, when herein the terms “beads” or “solids” are used in relation to particles of the granular material, this may also refer to liquid particles and/or particles comprising a liquid. This may be understood from the context. Yet, physically liquid particles may be composed of liquid. The particles may in embodiments further deform (though retaining their physical borders/remaining discrete). The constituents that compose granular material are large enough such that they especially are not subject to thermal motion fluctuations. Therefore, it is generally indicated that a lower size limit for granules/particles in granular material is about 1 μm. In some sense, granular materials do not constitute a single phase of matter but have characteristics reminiscent of solids, liquids, or gases depending on the average energy per granule. However, in each of these states granular materials also exhibit properties which are unique.

Granular materials may exhibit a wide range of pattern forming behaviors when excited (e.g. vibrated or allowed to flow). Granular materials under excitation can be thought of as an example of a complex system. The granular material is a complex system that may exhibit nontrivial transitions between the static, the quasi-static, and the dynamical states. Unlike most other solid or liquid materials, the tendency of a compacted dense granular material is to dilate (expand in volume) as it is sheared. This occurs because the granules in a compacted state are interlocking and therefore do not have the freedom to move around one another. When stressed, a lever motion may occur between neighboring granules, providing a bulk expansion of the material. On the other hand, when a granular material is in a loose state it may compact (and may become “jammed”). The behavior especially allows printing or extruding material that is in a jammed state (and may flow and dilate when forced to a nozzle or needle).

Granular systems are known to exhibit jamming and undergo a jamming transition which is thought of as a thermodynamic phase transition to a jammed state. Jamming is the physical process wherein the viscosity of some mesoscopic materials, such as granular materials, but also e.g. glasses, foams, polymers, emulsions, and other complex fluids, increases with increasing particle density. The jammed state is reached for an infinite viscosity, wherein the granular material is able to resist a finite shear stress without flow and thus becomes a solid. The jamming transition is a reversable transition and reflects a type of phase transition with similarities to a glass transition. Granular materials in a jammed state may be self-supporting. Granular material may further show self-healing properties. A minimal dilation and/or dilution of a jammed granular material may result in self-healing properties (of the granular material).

The (plurality of) granular material(s) described in relation with the construct may in embodiments have a (number averaged) particle size in the micrometer and/or millimeter range. The (number averaged) particle size may especially be at least 1 μm, especially at least 3 μm. In specific embodiments, the particle size is equal to or smaller than 3 mm, such as equal to or smaller than 1 mm. The particle size may especially be selected to be equal to or smaller than 250 μm and especially at least 3 μm, such as at least 5 μm, especially 10 μm, even more especially at least 25 μm. The term “particle size” especially relates to a number averaged particles size. Furthermore, for non-spherical particles, the size may be defined by the Sauter mean diameter, also indicated by d₃₂. In specific embodiment, the construct comprises (particles comprising) a plurality of particle sizes. Different compartments of different granular material may comprise particles with respective different particle sizes. By configuring compartments comprising particles with particle size that differ between the compartments, a gradient in a height of a channel formed between the particles in the interstitial cavity (see below) may be provided.

A total number of compartments is not limited. The total number of compartments may e.g. be at least 10, such as at least 100, or at least 1000, or even more. Essentially, a compartment may comprise a single particle, or a plurality of particles. Hence, in embodiments, a discrete difference between adjacent compartments may hard to observe. In embodiments the compartments (or particles) may define as smooth gradients of the material types. In further embodiments, one or more of the compartments may not be distinct.

Further, the invention is not limited to a specific particle shape. The particle may comprise any arbitrary shape. The particle may e.g. be spherical, or non-spherical such as ellipsoid, or sphenoid. In embodiments, the particle may have an aspect ratio higher than 1, for example, comprise an elongated shape, such as fiber-shape, a rod-shape, or a ribbon-shape. In embodiments, the shape of the particle (or of different particles in the granular material) is a combination of the aforementioned shapes. The particle may further comprise a specific architecture, such as a microfabricated architecture and/or a microfabricated cage. The particle may further have a smooth surface, or in other embodiments have a random or organized surface roughness, including a (determined) micro-topography.

The particle may be a solid particle. The particle may be a (dry) hollow particle and/or a hollow particle with a liquid shell. The particle may be a liquid filled particle. Additionally or alternative, the particle may be a particle with multiple (particle) compartments, such as a Janus particle. In embodiments, the particle is a coated (hydrogel) particle. In yet further embodiments, the particle comprises a vesicle, for instance a liposome and/or a capsosome. The particle may further be a core-shell particle. Moreover, the particle may be selected from the group of a solid particle, a (dry) hollow particle, a hollow particle with a liquid shell, a liquid filled particle, a particle with multiple compartments, such as a Janus particle, a coated (hydrogel) particle, and a core-shell particle. The particle may further comprise one or more particles in the particle. For instance, a core-shell particle may comprise one or more further particles arranged in the particle, especially in the core of the particle. The particle may e.g. comprise one or more vesicles. The particle may further comprise a solid matter (i.e. no liquid or gaseous matter). The particle may further comprise a specific particle “class” of particles. The particle may e.g. be a hydrogel particle, a cell spheroid, a loaded particle (e.g. comprising a growth factor and/or a protein and/or a biological cell). Herein, these different classes, shapes, types, etc. may all be referred to by the terms “a property of the particle” or “a particle property (of the particle)”. The particles (of a granular material) may be selected based on one or more of the particle properties described herein. In further embodiments, the particles are wet (liquid comprising) particles.

The term “particle” may refer to a plurality of (different) particles. For instance in a phrase like “the particle of a first granular material”, the term may relate to a plurality of substantially the same particles. Yet, the first material may also comprise a plurality of different particles, e.g. comprising different size (see below). In a phrase like the particle of the first material type and “the particle of the second material type”, the particle property of the particle of the first material type may differ from the particle property of the second material type.

The term “vesicle ” is known to the skilled person and may relate to a structure within or outside a cell, consisting of liquid or cytoplasm enclosed by a lipid bilayer. Vesicles may be formed naturally. Alternatively, they may be prepared artificially, especially indicated as “liposomes”. Vesicles may also be core-shell particles. A capsosome may be prepared by combining liposomes and polymer capsules and may especially comprise a plurality of liposomes. The capsosome may also be a core-shell particle. The term “vesicle” may in embodiments, especially refer to one or more of a liposome and a capsosome. In embodiments, the particle comprises a lysosome and/or a capsosome. In further embodiments, the particle comprises one or more particles comprising a lysosome and/or a capsosome.

Further examples of particle properties of a particle (or a group or cluster or compartment of the particles) are a storage modulus, a loss modulus, a Young's modulus, a stiffness, a chemical composition, a deformability, a size, a (surface) charge, a payload fraction (i.e. a fraction of a payload of the particle), a crosslinking density (of e.g. polymers of the particle), a porosity (of the granular material), a toughness, a stress recovery (or relaxation) time, a thermo responsive property, a photo-sensitive property (such as the ability to be cross-linked by UV and/or visual light, etc.), building blocks (especially a type of polymer) of the particle. The particle may be a hydrogel particle, especially comprising a polymer. In embodiments, the particle property comprises a type of polymer and/or a polymer concentration. The particle may comprise a compound particle, such a (biological) cell-laden microgel, or nano-particle(or micro-particle)-laden microparticle (e.g., for release of actives). The particle may in embodiments comprise a solid and a liquid, especially a solid in a liquid.

Particles may especially be selected depending on the tissue to be cultured. The particle may e.g. comprise collagen for culturing smooth muscle cells or skin tissue rich in collagen. In embodiments the elasticity E-modulus (or Young's modulus) of the particle is in the range of 0.5-100 kPa, such as 0.5-50 kPa.

Furthermore, the particle property may be related to the plurality of particles (forming the granular material). As such a further particle property (or granular material property) may e.g. comprise the dispersity (of the particles in the granular material), e.g. being monodisperse (especially comprising a (characteristic) size (e.g. diameter) distribution having a coefficient of variation in size smaller than 10%, see below), polydisperse, biphasic, or polyphasic. Hence, particles of one material type may in embodiments comprise different sizes (in a particle size distribution). The particle property may further comprise the porosity, an electrostatic interaction (between particles of the granular material and/or relative to particles of another granular material), and an interstitial liquid viscosity (of liquid between particles of the granular material). In embodiments, a surface charge of particles of one of the material types and the surface charge of particles of another one of the material types have opposite values.

Many particle properties are described herein. In embodiments, the particle (of a determined (granular) material type) may especially be selected based on the size (and/or size distribution) of the particle. Additionally or alternatively, the particle may be selected based on the shape of the particle. The particle may e.g. in specific embodiments comprises a spherical particle. Additionally or alternatively, the particle may be selected based on the polymer concentration in the particle. Additionally or alternatively one or more of the particles may be selected based on the surface coating of the particle. Especially, the construct may be configured by controlling one or more of the particle size, the particle shape, the polymer concentration, and the surface coating of particles of determined material types (and/or compartments) in the construct.

The term “particle property” may in embodiments refer to a property of the particle in the granular material, and/or a granular material property. Moreover, a property of the granular material may especially be defined by a property of the particle (of the granular material). The particle(s) may especially be selected for providing a determined property of the granular material (type), even more especially to one or more compartments. Herein the term “property” in relation to an element such as a particle, may especially refer to a characteristic of the element. For instance “the particle property” may refer to “the particle characteristic”.

The particle may comprise a payload. The particle property may comprise a payload (property). The term “payload” especially refers to an amount of a functional (chemical or physical) component in or at (the surface of) the particle. The particle may e.g. be loaded with a biological element. Examples of such biological element are, e.g., a biological cell, an organoid, an embryoid (body). The biological element may e.g. be a protein or a peptide, or an oligonucleotide. The protein and/or peptide may comprise a growth factor. In further embodiments, the biological cell is a growth factor. Additionally or alternatively, the particle is loaded with a subset of smaller particles (nano and/or micro particles). Hence, the particle may be loaded with a cellular aggregate and/or an organoid. The particle may further be loaded with a catalyst, and/or a photo-initiator, and/or a magnetic load, and/or a synthetic enzymatic reactor. The particle may comprise a capsosome loaded in the particle.

The term “enzymatic reactor” refers to an artificial construct that contains and sustains enzyme(s), while allowing them to react with the environment; the construct is especially synthetic/artificially made with synthetic polymers. The enzymes may e.g. be loaded in liposomes which may be loaded in/attached to, and/or be part of capsosome (inside the particle), wherein the enzyme is able to communicate with the environment (external from the particle). The enzymatic reactor may function or may be considered as a catalyst. Hence, in embodiments the catalyst may comprise an enzymatic reactor. The biological element, and chemical factors such as catalyst, photo-initiator may also be referred to with the term “bioactive compound”. The payload may in embodiments comprise a bioactive compound. The payload may be arranged in the particle and/or at the surface of the particle.

The particle may further comprise an interpenetrating polymer network. In an interpenetrating polymer network at least one polymer is configured penetrating further material (especially at least one other polymer) of the particle. An interpenetrating polymer network may e.g. be a network of materials wherein at least one polymer intertwines with one or more of the other material(s) in the network.

Hence, in embodiments, the particle comprises a protein and/or a peptide. The protein or peptide may especially relate to a growth factor and/or function as a growth factor. The protein (and/or peptide) may in embodiments be produced synthetically (artificially). In embodiments a synthetic protein (and/or peptide) may function as a growth factor. In further embodiments, the particles may comprise one or more growth factors. The term “growth factor” may relate to a plurality of (different) growth factors. Also the terms “protein” and “peptide” may relate to a plurality of (different) proteins and peptides, respectively. The protein and peptide may be natural and/or artificially made. In embodiments, the bioactive compound comprises one or more growth factors selected from the group consisting of an epidermal growth factor (EGF), a fibroblast growth factor (FGF), a vascular endothelial growth factor (VEGF), a platelet-derived growth factor (PDGF), an angiopoietin (Ang), a transforming growth factor beta (TGFβ), a bone morphogenetic protein (BMP), a cytokine, and a hormone. Above, a non-limiting list of growth factors are given, especially growth factors related to vascularization. The bioactive compound, especially the growth factor is not limited to the given embodiments. The invention may be directed to any arbitrary growth factor (including artificial protein/peptide). The growth factor is especially selected based on the tissue to be engineered. Furthermore, (synthetic) proteins/peptides may have a function as growth factors. Although, these synthetic proteins or peptide may not always be classified as growth factors. Herein, however the term growth factor may also relate to such proteins/peptides. Hence the term “growth factors” and the like may refer to growth factors as well as to synthetic proteins/peptides that may function as a growth factor. Furthermore the term “bioactive compound” also refers to the synthetic proteins and peptides that may function as growth factors.

Especially, (the particles in) the material types mutually differ at least in (a type and/or presence of) a growth factor (in/off the material type). In further embodiments, different material types may comprise different biological cells and/or cellular aggregates. In yet further embodiments, the material types may differ in the presence of (a subset of) smaller particles contained within the particle, e.g. the presence (and or type of) of nano- and/or microparticles.

The granular material may be shear-thinning. The granular material may be either in a jammed or unjammed state. The shear-thinning behavior may allow a printing needle to be moved freely in granular material. The granular material may locally exhibit laminar flow around the moving needle, and the material may transition (back) into the jammed state after the needle leaves the location or comes to a rest. The granular material may further function as supporting material for printed material through the self-healing properties. The granular material may further comprise additional matter, especially arranged between and/or around the particles. Granular material may e.g. comprise a gas (e.g. air or an inert gas), a yield stress solid (which is soft and can also flow under shear), or a liquid arranged between the particles. The granular material may further comprise a further type of granules/particles such as a powder. The powder may e.g. also be arranged between the particles and/or surrounding the particles.

Material configured between the particles (such as a gas, a liquid, and a powder, or any other non-granular material) may herein also be referred to as “interstitial material”. The granular material may comprise an interstitial liquid. In further embodiments, the granular material comprises an interstitial gas and/or solid (e.g. a powder). Hence, in embodiments, one or more of the material types comprising granular material further comprises an interstitial material arranged between the particles. The interstitial material may comprise one or more materials selected from the group consisting of a gas, a liquid, and a solid (especially a powder). Interstitial cavities (volumes) between the particles may facilitate and/or control growth or biological function or behavior of cellular tissue in the construct. Interstitial cavities, especially properties of the interstitial cavity may define (a part) of the spatiotemporal information in the construct. Additionally, e.g., further properties of the material type, especially (other) physical and chemical characteristics, define the information in the construct.

The interstitial material may be selected for having specific interstitial properties, such as type of material. The interstitial material may comprise a gas, a liquid, a powder, or e.g. any combination of the afore mentioned types. Such combination may herein also indicated be as “complex interstitial material” or “compound interstitial material”. Interstitial properties are especially physicochemical properties of the respective interstitial material alone and/or in combination with the particles. Such properties are known and only some examples are given below to illustrate some relevant parameters.

Examples of interstitial properties of a liquid interstitial material are, e.g., a viscosity, a yield stress, a stress recovery time, a thermo responsive property, a photo-sensitive property, (e.g. related to cross-linking by UV or visual light, etc.), an electrostatic charge, a polymer concertation (all of the liquid), and a volume fraction (of the liquid) with respect to the interstitial (cavity) volume.

Examples of interstitial properties of a gaseous interstitial material are, e.g., a density, a diffusivity, a compressibility, an expandability, a potential for chemical interacting with other compounds. Examples of interstitial properties of a powdery interstitial material are, e.g., a grain (powder) size, a melting temperature, a sublimation temperature, a potential for interacting with the particle (particle-grain interaction), a particle-grain mixing time, a liquid content and a grain mixing time. Likewise, examples of interstitial properties of a complex interstitial material are combinations of the above given properties.

The powder (grain) size is especially selected to be smaller than the particle size (of the respective particle in the granular material).

In embodiments, the interstitial material may provide further shear-thinning properties to the granular material.

The liquid (of the interstitial liquid and/or comprised by the particle) may in embodiments be (or comprise) water or a physiological solution or a solution optimized for culture of living entities such as mammalian cells. The liquid may comprise a nutrient (solution). The liquid may in an embodiment comprise a cross-linkable liquid or solution, e.g., comprising an initiator or catalyst to induce a crosslinking reaction (between and/or in (compounds of) particles). The liquid may especially be selected for having a viscosity selected in the range of 0.5-10000 m·Pas, especially 1-1000 m·Pas.

In embodiments, the liquid comprises a cell culture medium and/or a cross-linkable liquid and/or a crosslinker solution. In further embodiments, the liquid (further) comprises a pH buffering solution and/or a solvent (such as an alcohol, especially ethanol), and/or a liquid comprising a polymer. The polymer may comprise functionalizable or cross-linkable moieties.

Examples of pH buffering solutions comprised by embodiments of the liquid are, e.g., phosphate buffered saline, a zwitterionic sulfonic acid buffering agent, (4-(2-hydroxy-ethyl)-1-piperazineethanesulfonic acid (HEPES), HEPBS (N-(2-hydroxyethyl)piperazine-N′-(4-butanesulfonic acid), CAPS (N-cyclohexyl-3-aminopropanesulfonic acid), N-cyclohexyl-2-hydroxyl-3-aminopropanesulfonic acid (CAPSO), and N-Cyclohexyl-2-aminoethane-sulfonic acid (CHES).

Hence, the construct especially comprises (spatially arranged) compartments, wherein each compartment may comprise a subset of the number of material types. In specific embodiments, each subset comprises no more than one of the number of material types. Moreover, in embodiments, any one of the material types defines one or more compartments. Any one of the material types may be present in more than one compartment. At least part of each of the material types may define a (respective) compartment of the (spatially arranged) compartments. One or more of the material types may in embodiments be comprised by more than one (spatially arranged) compartment spaced apart from each other. In embodiments, a plurality of compartments may have substantially the same composition (comprise the same material type). Adjacently arranged compartments may especially comprise material with different (chemical or physical) properties. In specific embodiments, at least two compartments comprise granular material. In further embodiments, at least one (further) compartment comprises non-granular material (see below).

The construct especially comprises compartmentalized granular material. In embodiments, at least 30 vol.%, such as at least 40 vol.% of the construct comprises compartmentalized granular material. Especially, at least 50 vol.%, such as at least 75 vol.%, more especially at least 90 vol.% of the construct comprises compartmentalized granular material.

The term “compartment” or “compartmentalized” may especially relates to spatially arranged locations and spatially arranged over locations. Alternatively, this may be indicated as “the construct comprises ((spatially) arranged) compartmentalized material”. The construct especially comprises a plurality of (different) compartmentalized materials. One or more (especially at least two) of the compartmentalized materials may comprise granular material. In further embodiments, at least one (further) compartmentalized material may comprise non-granular material (see below). The compartments may define determined regions of the construct, e.g. a region to grow a specific part of a tissue (see also below).

The construct may be provided by additive manufacturing. The construct may comprise a graded and/or layered structure. In embodiments, (at least one of) the material types (further) defines at least part of a patterning of the construct. The material types are especially not homogeneously mixed. Hence, the construct especially comprises compartments (that differ from each other in material type).

In further embodiments, at least one of the material types comprises, especially is a non-granular material. The non-granular material may, e.g., be a gas, a liquid, or a solid. In further embodiments, the non-granular material may comprise a combination of a gas and/or a liquid and/or a solid or a combination of more than one liquid, gas or e.g. solid. The non-granular material may comprise a yield stress fluid and/or colloidal particles. The non-granular material may in further embodiments comprise a porous material such as foam. The non-granular material may comprise a soft phase, especially comprising a tunable porosity. The non-granular material may be configured in between at least two granular materials (or two compartments). In embodiments, the non-granular material (especially comprising a porous material) may prevent a cell to move between said granular materials and especially may allow e.g. nutrients to migrate via the non-granular material.

The non-granular material may comprise a solid (hard material), such as a metal or a ceramic. A solid material may provide a (desired) minimal stiffness to (at least part of) the construct. The non-granular material may especially be configured in spaces defined by the granular material(s). Such space is especially absent of granular material. Moreover, the space especially comprises a volume being larger than a (smallest) particle volume (of granular material defining the space). Hence, in embodiments the granular material(s) define(s) one or more spaces or compartments in the construct and the non-granular material(s) is (are) arranged in the spaces or compartments. Hence, in embodiments, the granular material(s) in combination with the non-granular material(s) define the compartments of the construct.

As discussed above, additionally or alternatively, one or more of the material types comprising the granular material may further comprise a non-granular material and/or a powder, especially arranged between the particles as interstitial material.

The terms “non-granular material”, and “powder” may also relate to a plurality of (different) non-granular materials and/or a plurality of (different) powders, respectively. Furthermore, a material type (comprising a granular material) may further comprise a plurality of different non-granular materials and/or powders. Essentially, granular material may also comprise non granular material arranged in the interstitial spaces between the granules. Granular material may comprise particles and non-granular material.

In embodiments, the non-granular material(s) comprise(s) a liquid, e.g. water, a cell culture medium, a pH buffering solution and/or a solvent. The liquid may further comprise a cross-linkable liquid or a crosslinker solution, or e.g. a polymer. The polymer may especially comprise functionalizable or cross-linkable moieties.

Hence, in embodiments, the non-granular material comprises one or more liquids independently selected from the group consisting of water, a cell culture medium (or “cell growth medium”), a cross-linkable liquid, a crosslinker solution, a pH buffering solution, a solvent (such as an alcohol, especially ethanol), and a liquid comprising a polymer (especially comprising functionalizable or cross-linkable moieties). In further embodiments, the granular material) comprises one or more of the liquids described in relation with the non-granular material, especially configured between and/or surrounding the particles.

The material types mutually differ in at least one chemical characteristic and/or one physical characteristics. Chemical and physical characteristics are sometimes interrelated. Therefore, “physical and/or chemical characteristic” may also be referred to as “physicochemical characteristic” “physicochemical parameter”, “physicochemical property”, or (only) “characteristic” and/or “property”. Hence, the “physical characteristic and/or chemical characteristic” may especially refer to one or more of the (material) properties and/or characteristics of the material types described herein.

The construct may thus comprise compartments with different material types (and properties). Yet, some compartments may comprise the same material type. The construct may be compartmentalized. Especially, the (different) material types may define one or more of the compartments. The material types may be selected for providing an embedding bath, especially for enabling tissue engineering. The construct may have specific construct properties, such as the spatiotemporal information defined by the material types.

The construct and/or compartment(s) may have further specific construct (and/or compartment) properties such as a storage modulus, a loss modulus, and a stiffness of the construct (or compartment). Further examples of these properties of the construct and/or compartment are a particle volume fraction, an interstitial liquid viscosity, a Herschel-Bulkley behavior, a yield stress fluid behavior (of interstitial fluid in the compartment/construct), an evaporation rate of particle content (especially in a compartment/construct with interstitial gas), a grain/particle size and/or an electrostatic interaction (especially in a construct comprising powder as interstitial material).

In embodiments, the (different) material types may especially (mutually) differ in a particle characteristic (of the particle of the respective material type). Additionally or alternatively, the material types may differ in a payload characteristic (of the particle of the respective material type and/or of interstitial material). In specific embodiments the material types mutually differ in at least one characteristic selected from (i) the group of particle characteristics and/or (ii) the group of payload characteristics (especially of the particle).

Some examples of particle characteristics that may differ between the material types are e.g. a (characteristic) number averaged size of the particle, a shape of the particle, a stiffness of the particle, and/or a surface property of the particle. A further example of such particle characteristic may be a composition of the particle. Further examples are listed above in relation to the particle properties.

The payload characteristic that may differ between the material types may especially be a presence or absence and/or a type of a biological element configured in the particle and/or configured at the surface of the particle. Another relevant payload characteristic may be the absence and/or presence of (a subset of) smaller particles contained within the particle and/or a catalyst (within the particle), and/or a photo-initiator within or at the surface of the particle and/or and a magnetic load of the particle. Relevant payload characteristics may further comprise one or more of the payload characteristics described herein. The payload characteristic may relate to the particle and/or to interstitial material between the particles.

In embodiments, the (group of) payload characteristic comprises (at least) one or more of a biological element configured in the particle and/or configured at the surface of the particle, a subset of smaller particles contained within the particle, a catalyst (within the particle), a photo-initiator within the particle and/or configured at the surface of the particle, and a magnetic load of the particle. Additionally or alternatively, the (group of) payload characteristics may comprise a bioactive compound and/or an interpenetrating polymer network contained within the particle. In embodiments, the payload characteristic comprises a bioactive compound contained in the particle.

The bioactive compound may in further embodiments be provided (only) after/by providing a further material in the construct, such as by the method for manufacture of an engineering tissue described herein. Embodiments of the construct described herein especially relate to an embedding bath configured for tissue engineering. The construct (and its properties) is especially described in relation to an embedding bath before biological cells that may grow to form tissue are dispensed in it. Hence, the construct as such may not provide a tissue and especially may require a further material to be disposed in it to allow tissue growth. In embodiments, the construct is configured for receiving biological cells. In specific embodiments, the construct (initially, especially directly after being produced with the method described herein) especially does not comprise biological cells that may grow to tissue.

In specific embodiments, the group of payload characteristics consists of at least a bioactive compound, especially a protein, peptide, (growth factor,) oligonucleotide, or other bioactive compounds described herein, contained within the particle; a subset of smaller particles contained within the particle (e.g. one or more nanoparticles contained within the particle, one or more microparticles contained within the particle); a charge of the particle; a biological cell contained within the particle; a catalyst contained within the particle; a catalyst configured at a surface of the particle; a photo-initiator contained within the particle; a photo-initiator configured at a surface of the particle; a magnetic load of the particle; an interpenetrating polymer network contained within the particle; a synthetic enzymatic reactor contained within the particle; a synthetic enzymatic reactor configured at a surface of the particle; and a vesicle (such as a liposome or capsosome) contained within the particle.

The group of payload characteristics may in further embodiments (further) comprise a chemical factor contained within the particle and/or a biological element contained within the particle or configured at the surface of the particle (e.g. a cell contained within the particle, a cell configured at a surface of the particle, a cellular aggregate contained within the particle, a cellular aggregate configured at a surface of the particle, an organoid contained within the particle, an organoid configured at a surface of the particle, an embryoid contained within the particle, an embryoid configured at a surface of the particle).

Hence, in embodiments, the particles of (at least one of) the granular material(s) have a number average particle size in the range of 3-3000 μm, such as in the range of 10-1000 p.m, especially 3-250 μm, more especially 5-250 μm, such as 10-250 μm, especially as 25-250 μm. In further embodiments, the particles of the granular material (of at least one of the material types) are selected to have a size distribution characterized by a coefficient of variation equal to or smaller than 10%. The term coefficient of variation (CV) is known to the skilled person and refers to the ratio of the standard deviation of the size of the particles to the average size of the particles.

In embodiments, one or more of the material types comprises a material type selected from the group consisting of a polymer, a glass, a ceramic, and a metal. The material type may e.g. comprise a natural or a synthetic polymer and/or polymer network, especially a hydrogel.

Hence, especially the particles (of one or more of the material types) comprise a material selected from the group consisting of a polymer, a glass, a ceramic, and a metal. In specific embodiments, the particles comprise a natural or a synthetic polymer and/or polymer network, especially a hydrogel. In embodiments the first granular material may comprise an (especially easily deformable or compressible) hydrogel and another granular material may comprise ceramic or steel particles. In specific embodiments, particles of at least one of the material types comprising granular material comprise a hydrogel, especially are hydrogel particles.

Hydrogels and/or hydrogel particles may e.g. comprise one or more of (the compounds or solutes) agarose, agarose collagen conjugate, alginate, alginate-dialdehyde, Alginate-1 -ethyl-(dimethylaminopropyl) carbodiimide hydrochloride (EDC), Oxidized methacrylated alginate, gelatin, collagen, chitosan, poly-ethylene glycol, gelatin methacryloyl, fibrin, polyethylene glycol (PEG), poly-ethylene glycol diacrylate, poly-ethylene glycol dimethacrylate, poly(trimethylene carbonate), fibroin, and hyaluronic acid. The hydrogel may in embodiments comprise polysaccharides (as one or more of the compounds/solutes). These compounds may especially provide cytological compatible and biocompatible hydrogels. The compounds are especially polymers. These compounds may e.g. be present in a concentration selected in the range of 0.1-100% w/v, especially 0.25-50% w/v, such as 0.5-25% w/v (weight of the compound/solute in grams per 100 ml liquid (solvent, especially water) (in the particle)). The hydrogel particles may in embodiments comprise at least 0.25% w/v, such as at least 1% w/v, even more especially at least 5% w/v of the compound. In embodiments, the hydrogel may consist substantially only of one of the compounds. The hydrogel may e.g. substantially only consist of poly-ethylene glycol, such as PEG400, such as for at least 95% w/v, especially for at least 97% w/v, even more especially for at least 99% w/v. In further embodiments the concentration of the compound in the (hydrogel) particle is 50% w/v at maximum. Hydrogel particles comprising a high concentration of the compound may e.g. provide a high stiffness. Particles with a high stiffness may provide rather inert compartments.

In specific embodiments (the particle comprising) the hydrogel (and especially also one or more of the material types) comprises polysaccharides, agarose, alginate, alginate dialdehyde, Alginate-l-ethyl-(dimethylaminopropyl) carbodiimide hydrochloride (EDC), Oxidized methacrylated alginate, gelatin, collagen, chitosan, poly-ethylene glycol, gelatin methacryloyl, fibrin, poly-ethylene glycol diacrylate, poly-ethylene glycol dimethacrylate, poly(trimethylene carbonate), fibroin, and hyaluronic acid.

As may be understood from above, a difference between the properties of the particles in a first granular material and the properties of the particles in another granular material may be very large in embodiments. Yet, in further embodiments the difference may be very small. In embodiments, a Young's modulus of (one or more of) the particles may e.g. be in the range of 100 Pa-1000 GPa, such as 100 Pa-10 MPa, especially 100-100,000 Pa. Moreover in embodiments, the Young's modulus of the particles of a first one of the material types comprising the granular material relative to the Young's modulus of particles of another one of the material types comprising the granular material is in the range of 0.001-1000. In further embodiments, a ratio of the storage modulus of the particles in a first granular material relative to the one of a second granular material is selected in the range of 0.001-1000.

In further embodiments, the construct comprises a cartridge frame (“frame”) enclosing (the assembly of the number of material types of) the construct and/or is enclosed by a cartridge frame. Especially, the (free-standing) construct is enclosed by the cartridge frame. In embodiments a frame encloses the embedding bath. Hence, in a further aspect, the invention provides a construct as described herein enclosed in a cartridge frame (“frame”). In yet a further aspect, the invention provides a cartridge frame (“frame”) comprising a construct described herein. The frame may facilitate transport of the construct without changing the patterned information. Moreover, in specific embodiments, the frame may be configured for providing mechanical support to the construct after increasing the liquid content of the construct, e.g. to provide the construct being self-healing. This may facilitate the method in specific embodiments wherein the range of volume fractions v_{f} to provide the self-healing construct does not overlap with the range of volume fractions v_{f} to provide the self-standing construct. In further embodiments, the range of volume fractions of to provide the self-healing construct and the range of volume fractions of to provide the self-standing construct overlap. Hence, especially the frame does not need to provide mechanical support to the construct. The frame may in further embodiments, (also) facilitate the method of production of the construct (see below). The frame may further be configured to support the tissue engineering process. Natural tissues respond to mechanical stimuli, for example fluid flow, contraction, tension, electrical stimulation, pressure, etc. Providing these properties to the construct (via the frame) may further improve embodiments of the tissue engineering.

The construct may be provided in a cartridge frame. Hence, herein the term “exterior surface of the construct” may in embodiments refer to an exterior (external) surface of the (assembly of) the material types (e.g. in the frame). The exterior surface especially does not refer to an external surface of the cartridge frame. In embodiments, the exterior surface contacts the cartridge frame. Moreover, the term “self-standing” and the like in relation to the construct/embedding bath (or granular material) essentially refers to the property (self-standing) of the construct/embedding bath or granular material as such. Hence, a self-standing construct, etc. is essentially (already) self-standing without any cartridge frame or container that may support the construct. Especially, the granular material defining the exterior surface is self-supporting.

The frame may in embodiments comprise an inlet and an outlet for providing, e.g., a fluid to the construct, and especially providing a fluid flow through the construct enabling to perfuse the construct. The fluid may especially comprise one or more of the liquids described herein (e.g. in relation to the interstitial liquid). The fluid may further comprise a gaseous fluid, such as air or an inert gas or a mixture of gasses. The fluid may in embodiments, comprise cells, chemotactic moieties, labelling moieties, and/or drug molecules. The fluid and/or elements in the fluid may be diffused, and/or be steered magnetically through the embedding bath compartments (especially in the method for producing engineering tissue described herein). The inlet and outlet may comprise a (set of) terminal(s) or connector(s) configured for guiding a fluid flow (of the fluid) within/through the construct, especially perfusing the construct. The connectors may be configured for connecting a supply and/or discharge line to the frame (and to the material types). The connector may in further embodiments be configured for providing an electric stimulus to the construct (the material types). In specific embodiments, the construct may further comprise conducting elements, especially for transporting an electrical current to determined locations in the construct. The conducting elements may be configured in conducting contact with one or more of the terminals. The construct may in embodiments comprise magnetic particles and/or conducting particles (especially functionally connected to the terminal(s)).

In embodiments, the frame is transparent allowing to visually observe changes in the construct. The frame may be configured to be opened and closed for further modification or extraction of (part of) the contents within it. Additionally or alternatively, the frame may not completely enclose all of the material types. For instance, a (top) side of the construct (material types) may not be covered by the frame.

Hence, in embodiments, the frame comprises one or more terminals for providing one or more types of stimuli to (especially to at least one of the compartments (defined in)) the construct. The frame especially comprises one or more terminals for providing one or more types of stimuli to at least one of the compartments (of the construct). The (types of) stimuli may especially be selected from the group consisting of a fluid flow to and/or through the construct, especially through and/or to the (determined) compartment, a provision of a chemical component (to the construct, especially to the compartment), a provision of an electrical current (to the construct, especially to the compartment), a provision of a magnetic signal (to the construct, especially to the compartment) and a provision of a drug (to the construct, especially to the compartment).

In a further aspect, the invention provides a method for producing a construct, especially the construct described herein (“construction method”). The construct is especially self-supporting. The (construction) method comprises providing a number N of material types at a substrate (to provide the assembly of material types at the substrate), especially wherein at least one, especially at least two, of the material types comprises granular material (comprising particles). Especially, N is at least 2. The construction method especially comprises an additive manufacturing technique. In embodiments, (at least one of) the material types comprising granular material is provided at (and/or in) the substrate using an additive manufacturing technique to provide at least a part of an exterior surface of (the assembly of material types of) the construct. The (at least one) material type comprising granular material (being deposited (using the additive manufacturing technique)) may especially be in a jammed state (before and especially also after depositing) The material type comprising the granular material may especially be a bioink. In embodiments, (the material type comprising) the granular material is self-supporting. The material type comprising the granular material may be shear-thinning, especially allowing to print the material type. Further, the material comprising the granular material may be self-supporting before and after depositing it at the substrate. The material comprising the granular material may have a volume fraction of the particles in the granular material as described herein in relation to a self-supporting material.

The term “bioink” is known to the skilled person and especially relates to an extrudable synthetic or biological material or a mix of both, which may or may not contain biological cells. The material types provided at the substrate may especially be selected from the material types described herein. The substrate may have a flat surface. The substrate may be curved. Essentially, the substrate may have any arbitrary shape. The phrases like “providing the material types at the substrate” especially relate to providing (such as printing) the material type on (top of) the substrate.

Herein, the terms “depositing” and “providing” in relation to an element and a location, such as in the phrases “depositing a material at the substrate” and “providing a material at the substrate” may especially relate to an action that results in a direct and/or an indirect contact between the element and the location, such as between the material and the substrate. For instance, when a first material is already deposited at the substrate (and contacting the substrate) depositing the second material at the substrate may result in depositing the second material on top of the first material (thereby providing an indirect contact between the second material and the substrate).

In specific embodiments, at least two of the number of material types (are selected to) comprise (a respective) granular material and the at least two of the number of material types (comprising (the respective) granular material(s)) are deposited simultaneously and/or sequentially at the substrate (using the additive manufacturing technique).

The additive manufacturing technique may especially comprise (3D) printing of (one or more of the material types comprising) granular material, especially via a (respective) printing nozzle or needle. The granular material may behave like a filament during printing. The (material type comprising) granular material may in embodiments be deposited layer by layer. In further embodiments, the additive manufacturing technique further comprises extruding and/or injection molding of one or more of the material types in one or more spaces defined by one or more of the deposited material types. Material (types) being deposited using injection molding and/or extrusion may be non-granular material. Yet, also granular material may be deposited by injection molding and/or extrusion.

In further embodiments, the (construction) method further comprises depositing (independently from each other) at least one of the material types comprising (a respective) granular material at a pre-assemble substrate to provide one or more building elements comprising the respective material types with a prearranged shape (or geometry). Successively, the one or more building elements may be provided at (especially assembled at) the substrate. The (granular) material types may therefore especially be selected to be self-supporting. In embodiments, the one or more building elements may define at least part of the compartments (of the final construct). In embodiments, one or more building elements after assembling define the (final) construct. In further embodiments, before, after and/or during the provision of the building elements, further (additional) material types may (also) be provided at the substrate. The granular material (being deposited) may especially comprise a bioink, especially being printed at the substrate.

In further specific embodiments, the (construction) method further comprises a remodeling stage, especially following (and/or during) the provision of the material types at the substrate. The remodeling stage comprises forcing particles of at least part of the material types (or compartments) (comprising granular material) provided at the substrate to move relative to each other, especially wherein a spatial arrangement of the material types at the substrate is adjusted. Hence, especially in the remodeling stage, a configuration of the compartments may be adjusted. Forcing may e.g. comprise smearing and/or spreading one or more of the material types. As discussed above, the granular material may define spaces in the construct. During remodeling dimensions of the spaces may change and/or one or more of the spaces may be removed. Especially, dimensions of one or more of the compartments may change in the remodeling stage.

In embodiments, the substrate comprises a pre-shaped mold and/or a frame. Hence, the material types may in embodiments be provided in the mold (and/or) frame, especially at a bottom of the mold (and/or frame).The pre-shaped mold may comprise a specific shape supporting the remodeling. The pre-shaped mold may in further embodiments comprise the terminals and may define the frame of the construct.

In embodiments, especially wherein the construct is provided configured for being self-healing by changing the liquid content of the construct, the (construction) method further comprises changing the liquid content of the construct, wherein a self-healing construct is provided. In embodiments of the method, liquid is extracted from the construct (to change the liquid content and the vf). In further embodiments, liquid is added to the construct. The volume fractions of particles in the construct, especially in the granular material, may in embodiments be changed (controlled) by extraction or addition of liquid to the construct.

Additionally or alternatively a temperature of the construct may be increased and/or a salt (solution) may be provided to the construct for providing the self-healing (properties of the) construct. Hence, in specific embodiments, the granular material may be rehydrated and/or resuspended in order to allow 3D printing a further material within the construct (such as in the method for tissue engineering, see below).

By using the (construction) method of the invention especially a patterned or compartmentalized embedding bath (or precursor embedding bath) is provided.

In yet a further aspect, the invention provides a method for the manufacture of an engineered tissue (“tissue engineering method”). The tissue engineering method comprises (i) providing a construct defined herein or obtainable by the construction method of the invention, and optionally changing the liquid content of the construct to provide the construct, wherein the construct is self-healing, (ii) locally dispensing a further material in the (self-healing) construct, and (iii) growing (or culturing) the tissue from one or more biological cells in the construct. Growing is especially done in a culturing period. The term “growing” may refer to culturing.

In embodiments, the (provided) construct comprises (the) one or more biological cells. Additionally or alternatively, the further material comprises the one or more biological cells. Hence, the one or more biological cells may originate from the provided construct and/or from the further material. The further material may especially comprise a cell suspension. The further material may in further embodiments comprise cell aggregates or e.g. organoids. The further material may further comprise a liquid, a solid, and/or a granular material. The further material may (also) comprise a gas, an emulsion, or e.g. a foam. The further material may in embodiments comprise a hydrogel (hydrogel particles). The further material especially comprises particles. Such (hydrogel) particles may comprise properties as described above in relation to the granular material. Any of the materials of the further material may independently from the other(s) comprise a chemical factor such as described herein, e.g. a protein and/or a peptide and/or a growth factor. In specific embodiments, the growth factor belongs to the Vascular Endothelial Growth Factor group (VEGF1, VEGFF2, etc.), or the bone morphogenic protein group (BMP-1 or the transformation growth factor (TGF-α, TGF-β, etc.).

In further embodiments, the construct (also) comprises one or more of the protein/and or peptides, such as one or more growth factors. Additionally or alternatively, the 30 construct comprises the (or another) biological cell. In specific embodiments, the further material comprises one or more protein and/or peptides, such as growth factors. and especially (also) the biological cell. The further material may in embodiments comprise (the) cells in a cell culture medium. The growth factor may be any growth factor described above in relation with the particles (including protein/peptides functioning as a growth factor).

The term “further material” may especially refer to more than one (different) further material. More than one (different) further materials may especially (at least partly) be dispensed spatially and/or temporally apart from each other. For instance a first further material may comprise the cell (e.g. in a cell suspension) and being dispensed at a first location, and a second further material comprising growth factors for the cell may be dispensed at further locations in the construct, e.g. adjacent to the first location.

The further material may especially be dispensed in the construct using an injection device such as a printing nozzle, syringe or a hollow needle. The injection device may in embodiments have a largest width or diameter of 2 mm, such as 1 mm at maximum. In further embodiment said largest width or dimeter is 0.5 mm at maximum. Locally depositing the further material may also be regarded as ‘writing’ with the further material in the construct. The further material is especially dispensed (injected) in the construct along a spatial path. Because of the self-healing property of the construct, the construct may allow moving of the injection device in the construct and especially a possible trail that may have been made by the injection device may close and ‘heal’ again after passage of the injection device. The location where the further material(s) is (are) deposited may especially be selected based on the compartmentalization of the construct. The further material may e.g. be deposited in a determined compartment. Additionally or alternatively, the further material (or another further material) may be deposited at locations at the edge of one or more compartments of the construct. It may further be understood that the construct and the further material may be complementary or adapted for each other. As such, e.g. at least part of the growth factors or bioactive compounds may be configured in the construct (at determined locations/in specific compartments) and/or at least part of the growth factors or bioactive compounds may be part of the further material.

The biological cell(s) is (are) in embodiments selected from the group consisting of a cell, a cell aggregate, a spheroid, and an organoid.

The cell aggregate may in embodiments consist of a single cell type. In further embodiments, the cell aggregate may comprise a plurality of cell types. Likewise, the spheroid and/or the organoid may consist of a single cell type or may comprise multiple cell types. The cell (types) may in embodiments have a size in the range of 5-40 whereas a characteristic size of a spheroid or an organoid may especially be in the range of 10-500 μm or even more. Yet, also other sizes are not excluded, and cells of other dimensions may be present in the further material. In further embodiments, the one or more cells are selected from a mammalian cell, an insect cell, a fish cell, a plant cell, a yeast cell, and bacteria

The (one or more) cell(s) may in embodiments comprise one or more cell types selected from the group consisting of a stem cell, an induced pluripotent stem cell, an omnipotent stem cell, an adult stem cell (such as a bone marrow-, fat-, or blood-derived stem cell), a progenitor cell (including a skin, a neuronal, a vascular, and a muscle progenitor cell), a somatic cell (e.g. HUVECs (Human umbilical vein endothelial cells), smooth muscle cells, cardio myocytes, neural cells, beta cells, chondrocytes, and osteoblasts), and a genetically modified organism (including cell lines). The cell may in embodiments comprise a human aortic endothelial cell (HEAC) or a Madin-Darby canine kidney (MDCK) cell.

In specific embodiments, the further material comprises further particles comprising biological cells. Especially, a number averaged size of the further particles is selected larger than the number averaged size of the particles of the construct (the (patterned) embedding bath). This may prevent the further particles from moving (diffusing) between the particles of the construct. The further particles may be consumed, shrink, degrade or disintegrate under controlled stimuli, which allows the cells, tissue, spheroids or other biological entity present, to differentiate, expand, go quiescent, and/or proliferate, allowing the biological cell(s) to grow and especially extend between the particles thereby forming an artificial tissue architecture.

In further embodiments, a fluid flow is provided through the construct (during culturing), especially through the interstitial spaces between the particles (especially in determined compartments). In specific embodiments, a value (flow rate) of the fluid flow is dynamic in value and with a specified periodicity. The fluid flow profile may mimic the flow rate of the heart during development. The fluid flow profile may also mimic the flow profile of a disease case, such as hypertension.

In further embodiments, oxygen is provided to the culturing cells. Furthermore, the method may comprise controlling a temperature of the construct, e.g., in the range from 36-38° C., especially at about 37° C. Therefore, the method may further comprise arranging the construct in a culture incubator and maintaining suitable culture conditions, e.g. an atmosphere comprising about 5% CO₂ and 21% O₂ and a temperature described above.

The invention further provides an engineered tissue obtainable by the (tissue engineering) method described herein. The engineered tissue may, e.g., comprise a tissue selected from the group consisting of an organ, a subsystem of an organ or a combination of organs. The engineered tissue may in embodiments comprise (a part of) a skin, a heart or a liver. In further embodiments, the engineered tissue comprises (a part of) vascular network, lymphoid network, or nervous system. The engineered tissue may comprise an artery. Yet, in embodiments, the engineered tissue comprises a combination of (parts of) a liver and a gut, a heart and lungs, etc.

The tissue engineering method of the invention allows controlling cell behavior during culturing. An angiogenic response may be mitigated in a high resolution (especially defined by the particle size). The interstitial space between the particles may especially act as a patterning template for the cells being cultured. The intestinal space as well as nanoscopic pores that may be created or formed in the particles during the production of the construct as well as during the tissue engineering method provide a micro-porosity of the construct. The micro-porosity enables diffusivity of nutrients, cell growth and differentiation factors. The micro-porosity may further enable enhanced cell migration. During culturing, cells and/or cell aggregates may move through the interstitial space and other nanoscopic pores and extend, especially resulting in an interstitial space pattern (resolution) of a few micrometers. The resolution may depend on the shape, size and material composition of the particles (in specific compartments) of the construct. Compounds (cues) in the material types may be selected to trigger or signal determined biological activity, both spatially and temporarily. By selecting the materials types, degradation mechanisms in the construct may be controlled and release of compounds from the material types may be controlled in time. Additionally or alternatively supplementary material to trigger biological activity may be provided to the embedding bath, especially to specific regions in the construct via the terminals of the frame, during predetermined periods of culturing. The supplementary material may e.g. comprise a fluid, a cell culture medium and/or oxygen. The supplementary material may further comprise material described in relation with the further material.

The construction method of the invention enables to have control over locations where a determined material type is arranged. At the same time, the total of the provided material types acts as an embedding bath. The ability to 3D print in the embedding bath especially relies on properties of the material types. The construct is especially configured for comprising spatiotemporal information for culturing one or more of the tissues described herein.

The construct may e.g. be configured for culturing an artificial artery. To avoid an immune reaction, cells may preferably be derived from the subject needing the artery. Because of the different parts of the artery (from the inside to the outside comprising the endothelium, the smooth muscle cells, an external elastic membrane and connective tissue) the construct may be produced comprising a compartment (for defining an outer region of the artery) which is inert so that the artery has finite borders. For this compartment, inert particles may be used, e.g. hydrogel particles comprising agarose. A central compartment may be provided defining a central sacrificial region (eventually providing a cavity) in order to allow perfusion of the artery. In embodiments the central compartment comprises a granular material (type) comprising particles with a polymer having hydrophobic and hydrophilic parts e.g. a poloxamer (e.g. marketed as “pluronic”), gelatin particles, etc.. The construct may further have an endothelium area (compartment), proximal to the central compartment (central sacrificial region), in which endothelial cells can be printed. Proximal to the endothelial area in the construct, a smooth muscle cell area may be configured, for printing the subject's cells. This region or compartment may in embodiments be configured of material types comprising collagen, especially for enhancing the development time of smooth muscle cells. In further embodiments, a mural cell region (compartment) is configured at the outer region of the smooth muscle cell region, in order to facilitate culturing an external elastic membrane (of the artery). This region may especially be configured with a specific morphology gradient, allowing for mural cell mobility throughout the construct in order to have a faster migration rate. The region may comprise relative smaller particles. A smaller particle size may favor migration of the cell material over development. The dimensions and properties of the material types (e.g. materials, particle size and growth factors) of the regions may be configured based on the patient's anatomy. The regions described above may comprise a plurality of compartments, or may comprise a determined part of a compartment, e.g. comprising a location wherein the further material is deposited.

### BRIEF DESCRIPTION OF THE DRAWINGS

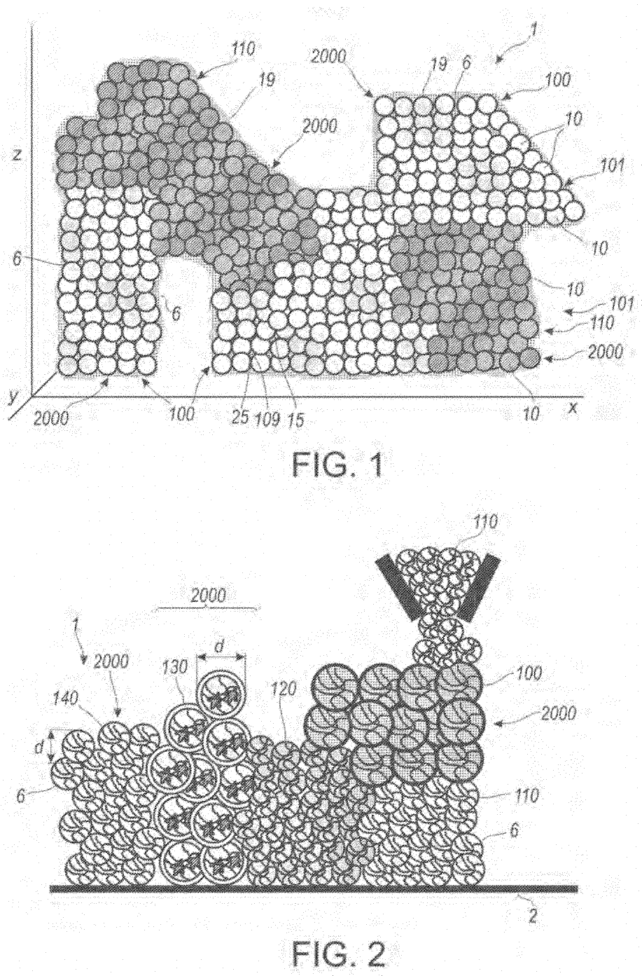

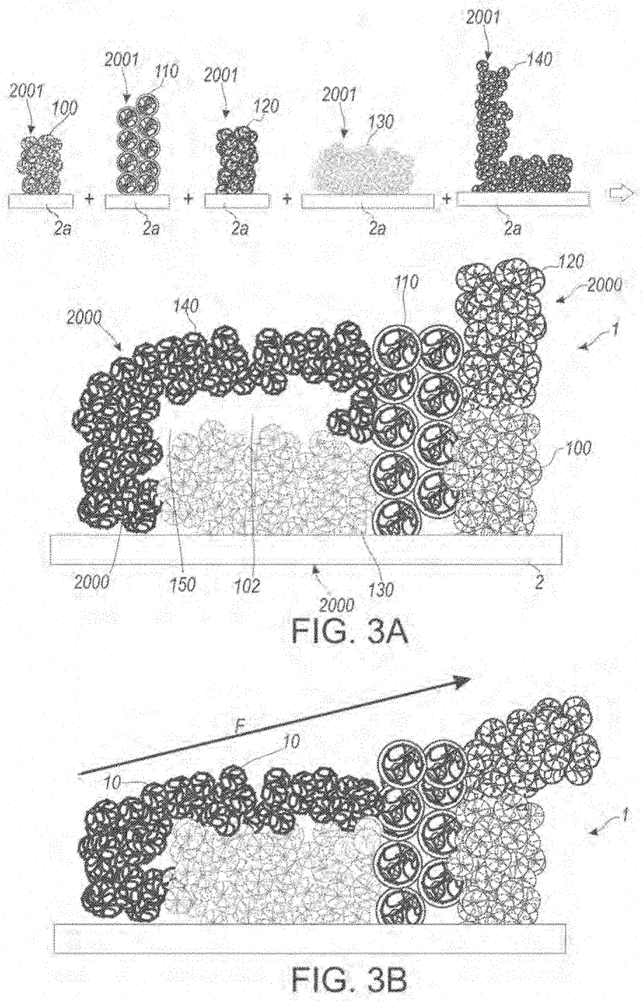

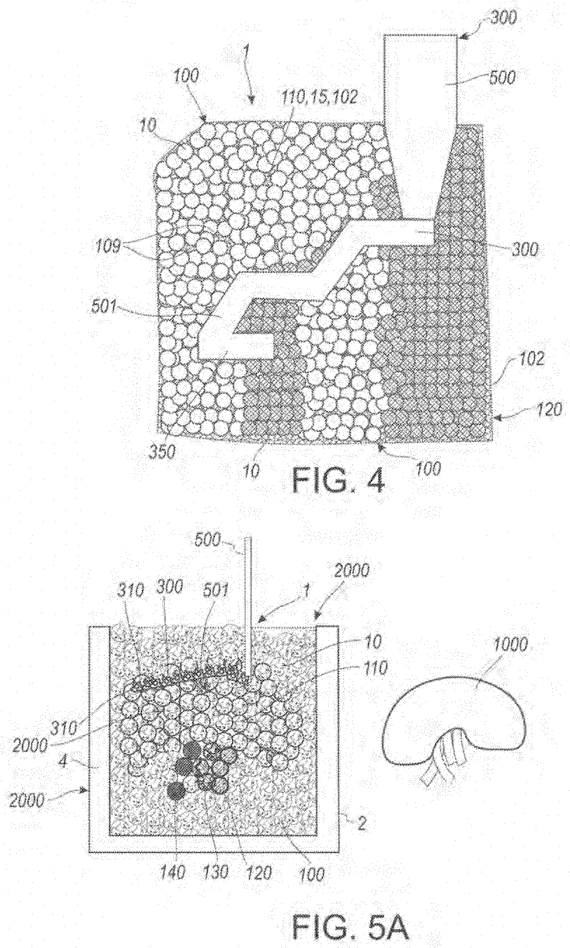

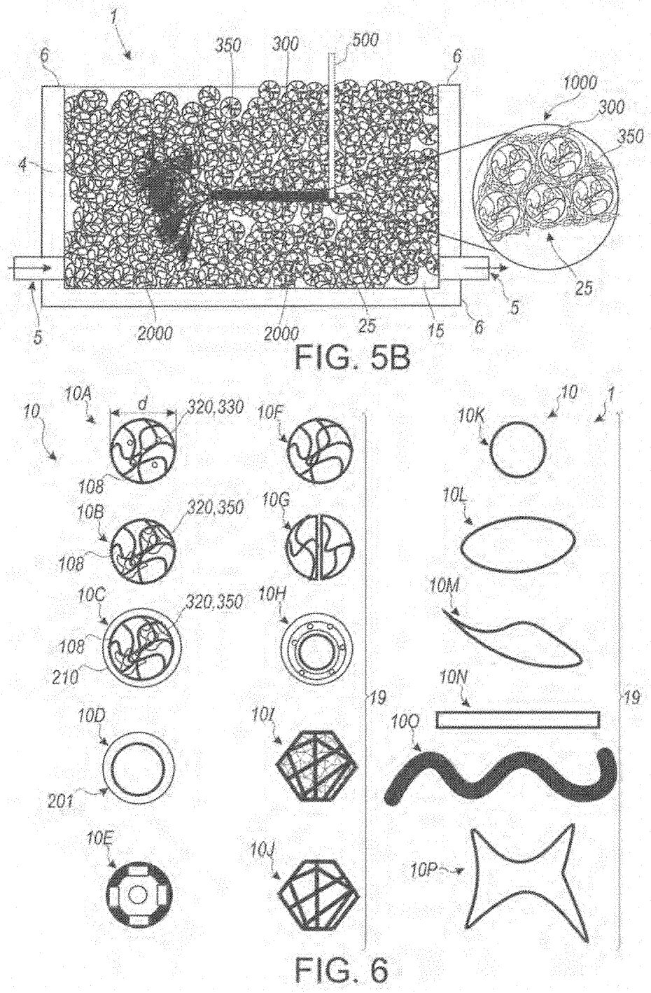

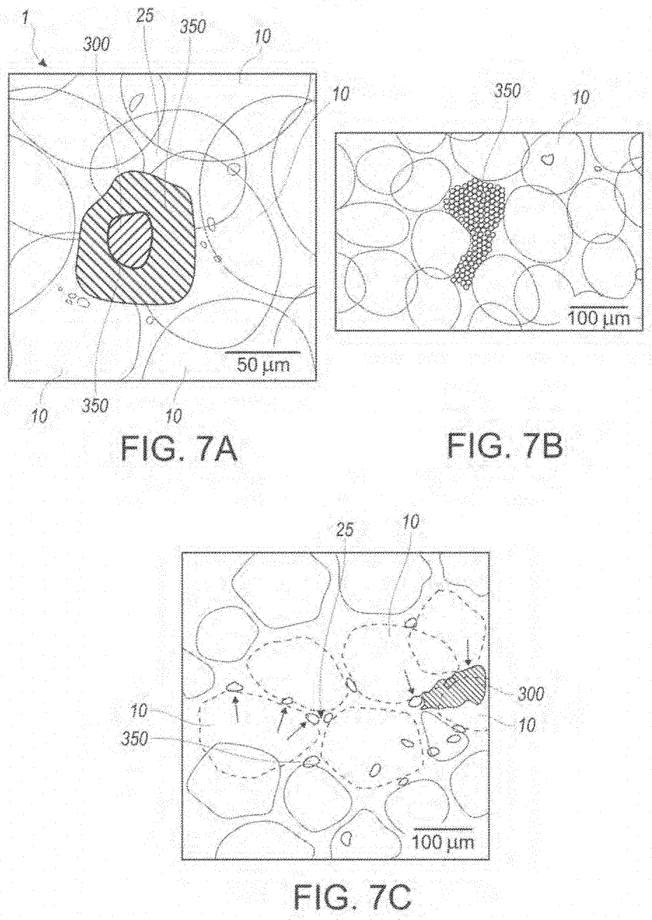

Embodiments of the invention will now be described, by way of example only, with reference to the accompanying schematic drawings in which corresponding reference symbols indicate corresponding parts, and in which: FIGS. 1 and 2 schematically depict some aspects of the construct and the method for producing the construct; FIGS. 3A and 3B schematically depict some further aspects of the method for producing the construct; FIGS. 4 and 5 schematically depict some further aspects of the invention; FIG. 6 depicts some embodiments of particles of the invention; and FIG. 7 depicts some further aspects of the invention. The schematic drawings are not necessarily to scale.

### DETAILED DESCRIPTION OF THE EMBODIMENTS

In FIG. 1 schematically an embodiment of the construct 1 of the invention is depicted. The construct 1 is depicted in 3D as is very schematically indicated by the X, Y, and Z axis, and indicated by the in the Y direction overlapping particles 10. For simplicity reasons, the depicted embodiment comprises only two material types 100, 110. Both of the material types 100, 110 comprise granular material 101. The granular material 101 of the material types 100, 110 comprises a plurality of (different) particles 10. The granular material 101 at least defines an exterior surface 6 of the construct 1. The construct 1 may further be self-healing. The construct 1 is especially self-supporting, especially indicating that the construct substantially will not lose its shape under its own weight. Hence, the (3D) shape depicted in the figure will substantially last for an extended period of time. The extended period of time may be in the range of minutes, hours, days, weeks or even more. In such extended period of time the dimensions, especially the size/dimensions of the external surface 6 may only change a few percent, especially less than 10%, such as less than 5%, even more especially less than 2%, or no more than 1% (relative to its initial dimensions, especially when being produced). This extended period of time is especially achieved in ambient conditions where the volume fraction (v_{f}) of the particles may not change, for instance by preventing evaporation of the interstitial liquid.

The construct is further especially (also) self-healing. The self-healing capacity may allow 3D printing a further material 300, e.g. comprising a biological cell 350, in the construct 1, see e.g. FIG. 4 and FIG. 5A. The volume fraction (v_{f}) of the particles 10 in the granular material 101 may especially be selected high enough to allow printing the granular material 101 and provide a self-supporting construct 1. Further, the volume fraction of the particles 10 in the granular material 101 may be selected to be low enough to provide the self-healing properties of the construct 1. The range in which the volume fraction is selected may depend on further physical parameters of the granular material 101 and/or the particles 10, such as the stiffness/elasticity of the particles 10 and e.g. the size distribution of the particles 10 in the granular material 101. Especially, the selected volume fraction (v_{f}) is larger than the random close packing of identical non-deformable spheres (64% v/v), especially larger than the densest packing of identical non-deformable spheres (74%), such as at least 80% (v/v), or at least 85% v/v. In embodiments the volume fraction of the particles in the granular material is selected in the range of 80-90% v/v to provide a self-healing and especially self-standing construct 1. In further specific embodiments, the construct 1 is configured for being self-healing by changing a liquid 15 content of the construct 1, especially by changing the v_{f} of the particles 10 in the granular material 101.

The different material types 100, 110 of the construct mutually differ in at least one characteristic 19, especially a physical characteristic and/or a chemical characteristic. In embodiment comprising more than two material types 100, 110, . . . , the types 100, 110, . . . may also differ in at least one such characteristic 19. For instance, the material types 100, 110, 120, 130, 140 depicted in FIG. 2 also mutually differ in at least one characteristic 19 selected from the group consisting of a physical characteristic and a chemical characteristic. The material types 100, 110, 120, 130, 140 may e.g. differ in an average size d of the particle 10 of the material type 100, 110, 120, 130, 140. They may further e.g. differ in a material of the particle 10, a stiffness of the particle 10, or 15 a liquid in the interstitial cavities 25 between the particles 10. They may further e.g. differ in a size distribution over the material type 100, 110, . . . .

Herein, the . . . . such as in phrases like “more than two material types 100, 110, . . . . ”

etc. refer to the (optional) further material types (e.g. 120, 130); and that the material types at least comprise material type 100 and 110.

The construct 1 is compartmentalized. The construct 1 especially comprises (or is made up of different) compartments 2000. A subset of the material types 100, 110, . . . may define or be comprised by such compartment 2000. The embodiment of FIG. 1 shows four (discrete) compartments 2000, wherein two compartments 2000 comprise the first material type 100, and two other compartments 2000 comprise the second (granular) material type 110.

It is noted that the granular material 101 type 100 may (exclusively) consists of particles 10 (see e.g. FIG. 2). Yet, in further embodiments, the granular material 101 consists of particles 10 and one or more other materials, e.g. a liquid 15, especially arranged in the interstitial cavities 25 between the particles 10 (see e.g. FIG. 1). Furthermore, in embodiments, granular material 101 of a first compartment 2000 may further comprise such liquid 15, whereas granular material of a further compartment 2000 of the same construct 1 substantially only comprises particles 10. Hence, in embodiments one or more of the material types 100, 110, . . . comprising granular material 101 further comprises an interstitial material 109 arranged between the particles 10, especially in the interstitial cavities 25. Such interstitial material 109 may especially comprise one or more materials selected from the group consisting of a gas, a liquid 15, and a powder.

Although not depicted in FIG. 1 or FIG. 2, not all material types 100, 110, . . . of the 30 construct 1 necessarily comprises granular material 101. For instance, one or more of the material types 100, 110, 120, . . . of the construct may comprise non-granular material 102. The embodiment schematically depicted in FIG. 3A at the bottom, e.g. comprises a space defined by two granular material types 130, 140 that comprises a material type 150 comprising a fluid such as a gaseous fluid, a liquid 15 or an emulsion as an example of a non-granular material 102. In other examples, e.g. the non-granular material 102 comprises a solid. Also these non-granular materials 102 may define /be comprised by a compartment 2000 of the construct 1. A solid compartment 2000 (a compartment 2000 comprising a solid material) may e.g. provide an additional stiffness to the construct 1. Especially, the further material 300 is provided in the granular material 101.

It is further noted that the numbering of the material types 100, 110, 120, 130, etc. are merely used to indicate different material types 100, 110, . . . The reference numbers of these material types 100, 110, 120, 130, etc. do not refer to the type, i.e. granular 101 or non-granular 102, of the material type 100, 110, . . . . Hence, in a first embodiment, material types 100, 110, 120, 130, and 140 may comprise granular material 101 and material type 150 may comprise non-granular material 102 (like FIG. 3A at the bottom). In a further embodiment, material types 100, 120, and 140, for instance comprise non-granular material 102, and material types 110, 130, and 150 comprise granular material 101. In FIG. 4, e.g., material type 110 configured in a space between particles of (granular) material type 100 is a non-granular material 102.

The embodiment of the construct 1 in FIG. 2 is especially (being) produced using an embodiment of the method of the invention as is schematically illustrated at the top side of the figure wherein (a part of) one of the material types 110 (at this stage) is provided, especially printed in 3D, at the substrate 2. It is noted that because already a number of material types (at least party) 100, 110, 120, 130, 140 are deposited at the substrate 2, the presently provided (part of the) material type 110 (only) indirectly contacts the substrate 2. Herein, providing a material type 100, 110, . . . at the substrate 2 refers to providing it such that it may directly or indirectly contact the substrate after providing it 100, 110 to the substrate 2. It is further noted that the figure depicts an embodiment, wherein the material type 110 is provided in two periods of time to the substrate 2. As such, at least two of the compartments 2000 of the final construct 1 may comprise the material type 110.

In the method, a number (especially at least 2) of material types 100, 110, . . . is provided at the substrate 2, especially wherein at least two of the material types 100, 110, . . . . comprise granular material 101, see e.g. FIG. 2 wherein five material types 100, 110, 120, 130, 140 comprise granular material 101. At least one of the material types 100, 110, 120, 130, 140 is provided at the substrate 2 using an additive manufacturing technique. As such at least part of the exterior surface 6 of the construct 1 is provided. The additive manufacturing may comprise 3D printing of the material type. In specific embodiments all material types 100, 110, . . . comprising granular material 101 are 3D printed. The substrates 2 of the embodiments in FIGS. 1 and 2 (as well as the pre-assemble substrate 2a depicted in FIG. 3A) are flat substrates, especially on top of which the granular materials 101 are deposited. The granular material 101 may keep its shape (while only being supported at the bottom by the support 2) because of its self-standing capacity.

The material types 100, 110, 120, may all be provided or deposited in one run at the substrate 2. Yet in embodiments at least one of the material types 100, 110, . . . comprising granular material 101 is initially provided at a pre-assemble substrate 2a, thereby providing one or more building elements 2001 comprising the respective material type 100, 110, . . . with a determined shape. Successively, the one or more building elements 2001 may be provided (as a building elements 2001/a compartment 2000) at the substrate 2, as is schematically depicted in FIG. 3A. One or more of the (at the substrate 2) provided material types 100, 110, . . . may further provide space, especially wherein successively a further material type 100, 110, . . . is provided.

Furthermore, in embodiments, the method may further comprise a remodeling stage as is depicted in FIG. 3B. In the remodeling stage particles 10 of at least part of the material types 100, 110, . . . provided at the substrate 2 may be forced to move relative to other particles 10 (of the same material type 100, 110, . . . and/or another material type 100, 110, . . . (especially relative to each other). As such, a spatial arrangement of the material types 100, 110 at the substrate 2, and e.g. also of the compartments 2000 is adjusted.

For providing a construct 1 to be free standing while also being able to function as an embedding bath (potentially after changing the volume fraction), especially a combination of characteristics of the particle 10 and/or of the granular material 101 seem relevant. The volume fraction of should be high enough to provide the self-standing capacity. The volume fraction of is especially selected above that of a random close packing (approx. 64% v/v) and even more especially above the maximum packing of non-deformable spheres (approx. 74% v/v). The volume fraction of may especially be in the range of 75-95% v/v, such as in the range of 75-90% v/v. Further, it is hypothesized that the particles 10 should be elastically deformable. In order to achieve a volume fraction of of over 74%, the particles 10 may especially be selected for being deformable. Especially, the deformation is elastic and not plastic. Plastic deformation may facilitate particle aggregation and may in embodiments prevent functioning as an embedding bath.

Further, having granular material 101 comprising particles 10 that may be free-sliding in along other particles 10 and especially are non-sticky may further facilitate the printing of further material 300 in the granular material 101 and the self-healing of the granular material 101. A motion of the injection device 500 or of a first particle 10 in the preferably does not drag another particle 10 with it, for example by particle-particle adhesion forces. Further, smooth sliding between particles 10 may be facilitated by minimizing of friction between the particles 10.

A further relevant parameter may be an extend of attraction between the particles 10. Minimizing the attraction between the particles 10 may facilitate free movement of the particles 10 relative to each other. Attraction of particles 10 may result in aggregation and/or in particles 10 that are not able to move freely with respect to each other when being sheared (e.g. when injecting a further material 300 in the embedding bath). In embodiments, particles 10 may be selected that show no interaction between particles 10 or even show repulsion between particles 10.

Furthermore, the granular material is especially shear thinning. The granular material further may show (especially complete) recovery after being sheared. These last effects may especially facilitate printability of the granular material. The factors may further facilitate the free standing and in embodiments also the shelf-healing of the construct

In embodiments (e.g. depicted in FIG. 2) the material types 100, 110, 120, 130, 140, . . . comprising granular material 101 may mutually differ in at least one characteristic 19, such as selected from the group of particle characteristics and/or payload 320 of the particle 10. The group of particle 10 characteristics may e.g. comprise of a characteristic number averaged sized of the particle 10, a shape of the particle 10, a stiffness of the particle 10, a composition of the particle 10, and a surface property of the particle 10. The particle 10 may e.g. comprise a coating 210. The particles 10 of the granular material 101 may in specific embodiments e.g. have a number average particle size d selected in the range of 10-250 μm. The particles 10 of different material types 100, 110, 120, 130, 140 especially differ in average particles size d. For clarity reasons all particles 10 of any of the material types 100, 110, 120, 130, 140 are spherical (globular) and have the same size d. In further embodiment, this may be different. In specific embodiments, at least one of the material types 100, 110, . . . comprises monodispersed particles 10, and especially the particles 10 of said material type 100, 110, . . . have a size distribution characterized by a coefficient of variation CV equal to or smaller than 10%. Further, particles 10 of at least one of the material types 100, 110, 120, 130, 140 may e.g. comprise a hydrogel (particle). Some further of examples of the properties 19 of the particle 10 are further depicted in FIG. 6 (see below).

The material types 100, 110, 120, 130, 140 may additionally or alternatively (also) mutually differ in a characteristic 19 from the group of payload 320 characteristics. Examples of payload 320 characteristics are e.g. a bioactive compound 330 contained within the particle 10, a subset of smaller particles 10 contained within the particle 10, a charge of the particle 10, a biological cell 350 contained within the particle 10, a catalyst contained within the particle 10 or configured at a surface of the particle 10, a photo-initiator contained within the particle 10 or configured at the surface of the particle 10, a magnetic load of the particle 10, an interpenetrating polymer network contained within the particle 10, and a vesicle 201 such as a liposome or capsosome comprised by the particle 10.

The bioactive compound 330 may herein especially refer to a biological element such as a biological cell 350, an organoid, an embryoid (body) or a protein or a peptide, especially a growth factor, or an oligonucleotide; or to a chemical factor such as catalyst, photo-initiator. Optionally the protein and/or peptide comprises a synthetic proteins and/or peptide that may function as a growth factor.

In specific embodiments, one or more of (especially the particles 10 of) the material types100, 110, 120, 130, 140, is be loaded with one or more growth factors such as an epidermal growth factor (EGF), a fibroblast growth factor (FGF), a vascular endothelial growth factor (VEGF), a platelet-derived growth factor (PDGF), an angiopoietin (Ang), a transforming growth factor beta (TGFβ), a cytokine, a hormone, a bone morphogenic protein (BMP), a cytokine, and a hormone.

In FIG. 5B, the construct 1 is (at least partly) enclosed in a cartridge frame 4. In the given embodiment, the cartridge frame 4 comprises two terminals 5 for providing one or more types of stimuli to at least one of the compartments 2000 (defined in the construct 1). Examples of types of stimuli that may be provided to at least part of the construct 1, or to one or more specific compartments 2000 are e.g. a fluid flow through the compartment 2000, a provision of a chemical component to the compartment 2000, a provision of an electrical signal to the compartment 2000, a provision of a magnetic signal to the compartment 2000, and a provision of a drug to the compartment 2000.

FIG. 6 schematically depicts some characteristics 19 of the material types 100, 110, . . . , especially of the particles 10 of the material types 100, 110, .. (comprising granular material 101). Particles 10A, 10B, 10C e.g. depicts hydrogel particles. In the depicted embodiments, these hydrogel particles are all loaded. i.e. comprise a payload 320. In particle 10A indicated by some dots, e.g., schematically depicting a bioactive compound 330. Particles 10B and 10C both comprise a cell 350. The particles 10 do not need to be loaded. Particle 10F e.g. depicts a hydrogel particle without a payload 320. The particle 10 may further comprise a coating 210, see particle 10C, being an embodiment of a core-shell particle. The particle 10 may comprise or be a vesicle 201. Especially, a core-shell particle 10 may comprise one or more vesicle 201. A vesicle 201 may especially comprise or be a core-shell particle 10 especially being hollow with an enveloping layer, see particle 10D. Further, the core-shell particle 10H comprises a (loaded) capsosome, schematically indicated by the inner circle with the small circles arranged in it. The vesicle 201 may herein also be called capsule. The particle 10 may comprise a Janus particle 10G having a different composition at a first part of the particle 10 compared to the other part of the particle 10. Further, the particle 10 may comprise a complex particle 10E comprising a combination of aforementioned characteristics.

The particle 10 may be a microprinted cage as depicted by particle 10J or it may comprise a complete microprinted architecture depicted by particle 101. The particles 10 depicted by particles 10A to 10J show examples of what is also referred to herein as particle classes. Particles 10K to 10P further depict some particle 10 shapes, like spherical (or globular), ellipsoid, sphenoid, fiber (or fibrous), ribbon, and complex, for respectively particle 10K, 10L, 10M, 10N, 10O, 10P.

The construct 1 described herein and/or obtained using the method to produce the construct may especially have embedding bath properties. The construct 1 may thus be configured for locally supporting a further material 300 being provided into the construct 1. To allow provision of a further material 300 into the construct 1, the construct preferably is self-healing. The method to produce the construct may in embodiment further comprises changing the liquid content of the construct 1, especially to obtain or provide a self-healing construct 1.

In FIGS. 4, 5A and 5B schematically some aspects of the method for the manufacture of an engineered tissue 1000 from one or more biological cells 350 is depicted. In said method a construct 1 described herein is provided.

The construct 1 is especially (already) self-healing (and self-supporting). In embodiments, the liquid content of the construct 1 may be changed to provide the construct 1, wherein the construct 1 is self-healing. In embodiments, the volume fraction of the particles in the granular material of may be reduced, such as a few % to provide the self-healing capacity. In the self-healing construct 1 locally a further material 300 is provided/dispensed (especially via a dispensing path 501 very schematically indicated in FIG. 4). The further material 300 is especially selected to comprises one or more elements of (i) one or more biological cells 350, (ii) a protein and/or peptide and/or a growth factor, and (iii) a liquid and/or a solid. In embodiments, the construct before providing the further material, (already) comprises one or more biological cells 350. In other embodiments, the construct 1 does not comprise a biological cell 350 to grow the tissue 1000 from. Hence, in embodiments the further material 300 at least comprises the one or more biological cells 350. After dispensing the further material 300, the tissue 1000 may be grown or cultured from the one or more biological cells 350 arranged in the construct 1.

The further material 300 may be locally provided in the construct 1, such as via an injection device 500. The further material 300 may be provided in the construct 1 along a spatial path 501 and/or at a discrete location in the construct 1. As such the further material 300 may be provided at determined spatial positions in the construct 1, e.g. in or relative to one or more determined compartments 2000. The method may comprise providing/dispensing (the same type of or different types of) the further material 300 in the construct, especially at the same or at another location in the construct 1. For instance during a first period further material 300 comprising a biological cell 350 is dispensed in the construct 1 (see e.g. FIG. 5A). During another period, e.g. a cell growth medium may be dispensed in the construct 1. Hence the spatial path 501 not necessarily is a continuous path but may refer to multiple different spatial path 501. In embodiments, the further material 300 is dispensed continuously or interruptedly in time in the construct 1.

The one or more biological cells 350 is (are) especially selected from the group consisting of a cell, a cell aggregate, a spheroid, and an organoid. Moreover, the one or more cells 350 may in embodiments comprise a mammalian cell, or a fish cell, or an insect cell. In further embodiments, the cell 350 may comprise a plant cell, a yeast cell, or a bacterium. Furthermore, the one or more cells 350 may comprise a stem cell, an induced pluripotent stem cell, an omnipotent stem cell, an adult stem cell, a progenitor cell, a somatic cell, and/or a genetically modified organism.

In specific embodiments, the further material 300 comprises further particles 310 comprising the biological cells 350. In specific embodiments, a number averaged size of the further particles 310 is configured larger than a number average size d of the particles 10 of the construct 1, especially to prevent spontaneous movement of the biological cell 350 between particles 10 of the construct. In FIG. 5A, the further particles 310 have about the same dimension as the particles 10 of material type 110. It is further, very schematically and simplified depicted in FIG. 5A that the construct 1 comprises a plurality of compartments 2000 of different material types 100, 110, 120, 130 providing the cues to grow the tissue 1000.

In the construct different particles 10 are configured that may especially during the method for the manufacture of the engineering tissue be consumed by growing biological cells 350 and/or that may define a path for the biological cells 350 to grow. Moreover, the particles 10 may shrink, degrade or disintegrate under controlled stimuli e.g. provided via the terminals 5 or via the further material 300. The cells 350, tissue 1000, spheroids or other biological entity present in the construct may (further) as a result differentiate, expand, or e.g. go quiescent or proliferate. This again may result in grows of the cells 350 and especially in the extending of the cells 350 between the particles 10 thereby forming an artificial tissue 1000 architecture as is very schematically depicted in FIG. 5B showing an expansion of the cells 350 in the interstitial cavities 25 between particles 10 as one of the examples of phenomena taking place during cell culturing.

Hence, using the method, an engineered tissue 1000 may be obtained, schematically depicted in FIGS. 5A and 5B. Examples of the engineered tissue 1000 are e.g. an organ, a subsystem of an organ or a combination of organs.

In FIG. 7, some pictures of experimental results are schematically depicted, showing the addition of biological cells 350 to the embedding bath (construct 1) and the differential behavior of the cells 350 deposited in constructs 1 of different compositions. FIG. 7A depicts a further material 300 comprising a spheroid of human smooth muscle cells (SMC) 350 together with human umbilical vein endothelial cells (HUVEC) 350 embedded in a bath or construct 1 (after depositing) of monodisperse alginate spheroidal particles 10 with culture medium as the interstitial fluid 109 in the interstitial cavities 25 at day 0.

FIG. 7B schematically depicts the same formulation (at a lower magnification) after four days of culture. It can be seen that the cells 350 migrate through the interstitial cavities 25 in between the alginate particles 10 to some extent, but that there is limited attachment of the cells 350 to the particles 10.

FIG. 7C shows the result after depositing a further material 300 comprising a spheroid of SMC together with HUVEC as the biological cell 350 in an embedded bath 1 of monodisperse alginate spheroidal particles 10 that were functionalized with collagen in the interstitial fluid 109 in the interstitial cavities 25, after three days of culture. Due to the collagen coating 210 of the particles 10, the cells 350 can attach to and interact with the particles 10. The embedded (and deposited further material 300) cell spheroid 350 is visible as the dark structure at the right side of the figure. It was experimentally found that that there is extensive migration and elongation of the cells 350 over the particles 10, indicated by the small open spots. The cells 350 migrated and elongated along the particles 10 of which the outlines are depicted by the dotted lines. Some of the larger extensions are depicted with small open spots of which some are indicated with the arrows. It is noted that the image represents a two-dimensional image, and extensions in the third dimension over the surface of the particles 10 are not shown. Based on these experiments, it may be concluded that the migration of the cells 350 among others may be controlled/supported by the type of material types 100, 110, . . . . of the construct 1.

The term “plurality” refers to two or more. Furthermore, the terms “a plurality of” and “a number of” may be used interchangeably. The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” includes also embodiments wherein the term “comprises”means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of”) but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”. The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method respectively. The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

### EXPERIMENTAL

### Initial Demonstration of 3D Printed Patterned Granular Construct

Patterned constructs have been prepared using 3D printing of granular material. In one example a computer file defining a 3D checker-board profile with squares (fields) of 5×5 mm in square and 5 mm in height. The “closed” squares representing one color of the fields were printed, thereby defining open (i.e. not printed) squares representing the other color of the fields. The (printed) squares are connected (only) at the edges (during printing) as such forming a continuous system. The printing was done using a printing nozzle of 400 μm. To complete the design (construct), 12 layers are deposited on top of each other. The construct was printed using monodisperse circular spheroidal particles with a diameter of 110 μm±7 μm, consisting of 0.8% alginate-di-aldehyde-gelatin (ADA-GEL) +0.25% alginate. The interstitial fluid was phosphate buffered saline (PBS).

The final construct resembled the design and showed the same checker-board profile with the open squares and closed (printed) squares having substantially the same size. The packing density of the particles in the resulting construct, defined as the fraction of the volume that is occupied by particles instead of interstitial fluid (wherein the total volume is occupied with particles and the interstitial volume) is >74% v/v. This density is above the maximum packing fraction of balls, due to the deformable nature of this formulation of particles i.e. the particles deform to allow for more dense packing. The results show that the construct is self-standing and self-supporting, even when tilted at a 90 degree angle. A 24-hour test wherein the construct was immersed in additional fluid showed that the construct remained stable for at least 24 hours (longer periods were not tested) under immersed conditions.

### Demonstration of the Use of a 3D Printed Patterned Granular Construct as an Embedding Bath

Further experiments were performed to demonstrate the use and capability of the construct as embedding bath. A patterned construct was made by 3D printing granular material to study the effect of deposition of a further material. The printed construct consists of 4 distinct squares with a height of 5 mm being connected (during printing) to each other along the edges thereby forming a larger square of 2×2 of the distinct smaller squares. Given that a printing nozzle of 400 μm is used, 12 layers are deposited on top of each other. The construct was printed using monodisperse slightly elongated spheroidal particles with a feret diameter of 211 μm±7 μm for the long direction and 140 μm±7 μm for the short direction, consisting of 0.8% alginate-di-aldehyde-gelatin (ADA-GEL) +0.2% alginate, coated with poly-L-lysine (PLL). The interstitial fluid used was phosphate buffered saline (PBS). The packing density of the particles in the resulting construct, defined as the fraction of the volume of the particles relative to the sum of the volume of the particles and the volume of the interstitial fluid, was >74%. This is above the maximum packing fraction of balls, due to the deformable nature of this formulation of particles i.e. the particles deform to allow for more dense packing. Experimentally it was shown that a circle of further material (4% Xanthan gum complemented with blue food coloring) could be printed and maintained (embedded0 within the granular construct.

Before the further material was printed within the granular construct, a small amount of liquid (water containing 0.2 M CaCl₂) was added to the construct to lower the packing density of particles to <74%. With this specific formulation of particles, a lower packing density seemed to be needed to give the construct embedding bath properties. The (3D) location of the embedded circle was stable (in time) within the construct, and the construct itself has not been damaged by printing within it, showing that this granular construct portrays favorable embedding bath properties. Microscopic images of the granular construct after deposition of the further material showed the close packing of the granules and the presence of granules (construct particles) on top of the further deposited material. The region where further material has been deposited, remained stationary after deposition.

### Further Experiments General

### 3D Printing of the Granular Material

Granular devices/constructs of 100 μm, 150 μm, 200 μm, etc. 0.8% ADAGEL (1:1 ratio; alginate di-aldehyde/gelatin crosslinked hydrogel) containing a PLL coating (incubated in 0.1% PLL (Poly-L-Lysine) solution overnight to attain a thin coating of max 1.5 μm), or 0.3-0.8% Alginate PLL coated (incubated in 0.1% PLL (Poly-L-Lysine) solution overnight to attain a thin coating of max 1.5 μm) microparticles, that on high-volume fraction of can be 3D printed (behave as an ink, shear thinning) and exhibit self-standing behavior upon deposition on a substrate, have been prepared. The particles are able to deform and recover their initial structure after shear (elastic recovery of particles, system is self-healing). Indicative values of storage modulus while at rest are 10,000 Pa-100 Pa (material dependent). The printing of the ADAGEL-PLL particles has been achieved with nozzles that are 2.5× times the particle size. 3D printing is consistent at 4x Nozzle size compared to particle size (i.e. 400 μm nozzle, with 100 micron particles, 600 μm nozzle, with 150 micron particles, etc.).

### Embedding Bath Properties (Self-Standing)

Some printed self-standing high-volume fraction granular compositions can act as embedding baths directly after printing. For other compositions, adding a small liquid volume (water, cell medium, . . . ) on the ink deposit readily reduces the volume fraction of the device, to reach a state of self-standing embedding bath with self-healing properties. Based on the first experiments, a feasible window of operation for 1-2% ADAGEL PLL and 0.5%-2% ADAGEL 0.25% Alginate PLL granular compositions may be between 70%-80% volume fraction (v_{f}) .

### Embedding Bath Properties (Not Self-Standing)

It appeared that adding more liquid volume may completely unjam the device returning into an embedding bath which is not free standing, or upon further dilution to a liquid solution (resuspension of particles without agglomeration).

### Other Particle Compositions

Similar behavior has been observed by particles consisting of 0.5-2% ADAGEL, 0.8% ADAGEL 0.3-0.5% Alginate, Gelatin and Ag/Col formulations. Similar behavior is expected of gelatin methacryloyl (GelMA) particles. It has been possible to create devices with different compositions, concentrations, particle shapes, with and without coating. Interstitial space composition

If fibrinogen is added in the liquid phase of the suspension, upon the addition of liquid volume that contains fibrin, the whole granular device can be annealed.

If cells are added into the liquid volume, such as smooth muscle cells (SMCs), they can proliferate and populate the interstitial space thus annealing the macro volume and deforming it. This has been observed with a concentration of lmillion cells/ml after 1 week of culture in ADAGEL-PLL particles. This effect became even more pronounced when combined with thrombin/fibrin annealing.

### Further Experiments—Some Specific Results

Constructs/bioinks were prepared from 0.8% ADAGEL-PLL, particles with a particle size of about 45 μm.

First type of constructs/bioinks were obtained by straining the particle solution with a cell strainer to remove the liquid by gravity. The first constructs had a of of 65-70% and were not self-standing.

Second type of constructs/bioinks were obtained by straining the particle solution with the cell strainer wherein a pressure drop of 0.02 MPa for 30 seconds is applied to remove an extra amount of the liquid compared to the first constructs/bioinks. The second constructs had a of of about 75-80% and exhibited self-standing and self-healing properties. It is noted that the self-standing aspect is observed but sharp features/corners may not always be retained.

Third type of constructs/bioinks were obtained by straining the particle solution with the cell strainer wherein a pressure drop of 0.04 MPa for 30 seconds is applied to remove an extra amount of the liquid compared to the second constructs/bioinks. The third constructs had a of of about 80-85% and showed self-standing properties and sharp features of corners may retain in the construct. These constructs may further be sculpted with a spatula, which demonstrates the retention of the ability of the particles to move in relation to each other.

Rheological characterization of the three types ofjammed 0.8% ADAGEL-PLL bio-ink (particle size ˜145 μm) showed for all types shear thinning behavior: Changes of viscosity with shear rate ramping up for all three types of constructs/bioinks. A continuous decrease in viscosity with increasing shear rate indicated that the systems were shear thinning.

Yielding of the three types of ink was tested under oscillatory shear: The three types of Jammed bioink were sheared at frequency f=1 Hz and 0.01 to 200% strain amplitude. For all types G′<G″ at the lowest strain and G″>G′ at highest strain demonstrating that the tested bioinks became liquid like with increasing strain.

Next to the three types of constructs, a granular assembly of ADAGEL-PLL 100 μm particles with 7-14 μm 1%w/v microparticles in the interstitial space was prepared and experiments were repeated. The particles were colored to show their presence. The experiments demonstrated that aforementioned behavior/properties, are also possible when 1%w/v (color) particles are added in the liquid phase. The added solid, rigid, neutral charge, color particles that are significantly smaller by an order of magnitude from the hydrogel particles that comprise the granular assembly, do not seem to participate or affect in a significant manner the jamming/unjamming properties of the device but they only seemed to change the color (the color particles were red).

In yet a further experiment, high vacuum volume fraction samples (based on straining at a pressure drop of 0.04 MPa) were scooped onto a glass slide. Then shaped with a spatula, then cut in half with a spatula, and then one piece was stacked on top of the other and merges. Overhangs of the top piece over the bottom piece can be observed, demonstrating that the construct is free-standing, that the particles in the construct may move freely with respect to each other and that the construct may be remodeled and/or may be assembled from smaller constructs. This specific sample is 2% ADAGEL 150 μm particles. The particles could be resuspended in a liquid solution without the presence of agglomerates.

1. A construct (1) comprising a number N of material types (100, 110, . . . ), wherein N is at least 2, wherein at least two of the material types (100, 110, . . . ) comprise granular material (101) comprising particles (10), wherein the granular material (101) at least defines an exterior surface (6) of the construct (1), wherein the construct (1) is self-supporting, and wherein the construct (1) is (i) self-healing or is (ii) configured for being self-healing by changing a liquid (15) content of the construct (1); wherein the different material types (100, 110, . . . ) mutually differ in at least one characteristic (19) selected from the group consisting of a physical characteristic and a chemical characteristic.

2. The construct (1) according to claim 1, wherein the construct (1) is self-healing and wherein the granular material (101) defining the exterior surface (6) is self-supporting.

3. The construct (1) according to claim 1, wherein the construct (1) comprises compartments (2000), wherein each compartment (2000) comprises a subset of the material types (100, 110, . . . ).

4. The construct (1) according to claim 1, wherein the construct (1) has embedding bath properties, wherein the construct (1) is configured for locally supporting a further material (300) being provided into the construct (1).

5. The construct (1) according to claim 1, wherein one or more of the material types (100, 110, . . . ) comprising granular material (101) further comprises an interstitial material (109) arranged between the particles (10), wherein the interstitial material (109) comprises one or more materials selected from the group consisting of a gas, a liquid (15), and a powder.

6. The construct (1) according to claim 1, wherein the material types (100, 110, . . . ) comprising granular material (101) mutually differ in at least one characteristic (19) selected from the group of particle characteristics consisting of a characteristic number averaged average size (d) of the particle (10), a shape of the particle (10), a stiffness of the particle (10), and a surface property of the particle (10).

7. The construct (1) according to claim 1, wherein the material types (100, 110, . . . ) comprising granular material (101) mutually differ in at least one characteristic (19) selected from the group of payload (320) characteristics consisting of a bioactive compound (330) contained within the particle (10), a subset of smaller particles contained within the particle (10), a charge of the particle (10), a biological cell (350) contained within the particle (10), a catalyst contained within the particle (10) or configured at a surface of the particle (10), a photo-initiator contained within the particle (10) or configured at the surface of the particle (10), a magnetic load of the particle (10), an interpenetrating polymer network contained within the particle (10), and a vesicle contained within the particle (10).

8. The construct according to claim 7, wherein the bioactive compound (330) comprises one or more growth factors selected from the group consisting of an epidermal growth factor (EGF), a fibroblast growth factor (FGF), a vascular endothelial growth factor (VEGF), a platelet-derived growth factor (PDGF), an angiopoietin (Ang), a transforming growth factor beta (TGFβ), a cytokine, a hormone, a bone morphogenic protein (B1V11³), a cytokine, and a hormone.

9. The construct (1) according to claim 1, wherein the particles (10) of the granular material (101) have a number average particle size (d) selected in the range of 25-250 μm and wherein the particles (10) of the granular material (101) of at least one of the material types (100, 110, . . . ) have a size distribution characterized by a coefficient of variation equal to or smaller than 10%, and wherein particles (10) of at least one of the material types (100, 110, . . . ) comprising granular material (101) comprise a hydrogel.

10. A cartridge frame (4) comprising a construct (1) according to claim 3, wherein the cartridge frame (4) comprises one or more terminals (5) for providing one or more types of stimuli to at least one of the compartments (2000), wherein the types of stimuli are selected from the group consisting of a fluid flow through the compartment (2000), a provision of a chemical component to the compartment (2000), a provision of an electrical signal to the compartment (2000), a provision of a magnetic signal to the compartment (2000), and a provision of a drug to the compartment (2000).

11. A method for producing a construct (1), wherein the method comprises providing a number N of material types (100, 110, . . . ) at a substrate (2), wherein N is at least 2, wherein at least two of the material types (100, 110, . . . ) comprise granular material (101) comprising particles (10), wherein the granular material (101) is self-supporting, and wherein at least one of the material types (100, 110, . . . ) comprising granular material (101) is provided at the substrate (2) using an additive manufacturing technique to provide at least part of an exterior surface (6) of the construct (1).

12. The method according to claim 11, wherein the material types (100, 110, . . . ) comprising granular material (101) are in a jammed state.

13. The method according to claim 11, wherein the method further comprises depositing at least one of the material types (100, 110, . . . ) comprising granular material (101) at a pre-assemble substrate (2a) to provide one or more building elements (2001) comprising the respective material type (100, 110, . . . ) with a determined shape, and successively providing the one or more building elements (2001) at the substrate (2).

14. The method according to claim 11, further comprising a remodeling stage, wherein the remodeling stage comprises forcing particles (10) of at least part of the material types (100, 110, . . . ) comprising granular material (101) provided at the substrate (2) to move relative to each other, wherein a spatial arrangement of the material types (100, 110, . . . ) at the substrate (2) is adjusted.

15. A method for the manufacture of an engineered tissue (1000) from one or more biological cells (350), the method comprising:
providing a construct (1) comprising a number N of material types, wherein N is at least 2, wherein at least two of the material types comprise granular material comprising particles, wherein the granular material at least defines an exterior surface of the construct, wherein the construct is self-supporting, and wherein the construct is (i) self-healing or is (ii) configured for being self-healing by changing a liquid content of the construct, wherein the different material types mutually differ in at least one characteristic selected from the group consisting of a physical characteristic and a chemical characteristic, wherein the construct (1) is self-healing,
locally dispensing a further material (300) in the self-healing construct (1), wherein the further material (300) comprises one or more elements selected from the group consisting of (i) one or more biological cells (350), (ii) a protein and/or peptide and/or a growth factor, and (iii) a liquid and/or a solid, and
growing the tissue (1000) from the one or more biological cells (350) arranged in the construct (1);
wherein the one or more biological cells (350) originate from the provided construct (1) and/or from the further material (300);
wherein the one or more biological cells (350) are selected from the group consisting of a cell, a cell aggregate, a spheroid, and an organoid.

16. The method according to claim 15, wherein the further material at least comprises the one or more biological cells (350).

17. The method according to claim 15, wherein the one or more cells (350) are selected from a mammalian cell, a fish cell, an insect cell, a plant cell, a yeast cell, and bacteria, and wherein the one or more cells (350) comprise one or more cells (350) selected from the group consisting of a stem cell, an induced pluripotent stem cell, an omnipotent stem cell, an adult stem cell, a progenitor cell, a somatic cell, a genetically modified organism.

18. The method according to claim 15, wherein the further material (300) comprises further particles (310) comprising biological cells (350), wherein a number average size of the further particles (310) is larger than a number average size (d) of the particles (10) of the construct (1).

19. An engineered tissue (1000) obtainable by the method according to claim 15, wherein the engineered tissue (1000) comprises a tissue selected from the group consisting of an organ, a subsystem of an organ or a combination of organs.
